(19) United States
(12) Patent Application Publication
(10) Pub. No.: US 2025/0025510 A1
(43) Pub. Date: Jan. 23, 2025

(54) MULTISPECIFIC T CELL ENGAGER POLYPEPTIDES IN COMBINATION WITH T CELLS FOR CANCER THERAPY

(71) Applicant: THE BRIGHAM AND WOMEN'S HOSPITAL, INC., Boston, MA (US)

(72) Inventors: Khalid SHAH, Andover, MA (US); Filippo ROSSIGNOLI, Andover, MA (US)

(73) Assignee: THE BRIGHAM AND WOMEN'S HOSPITAL, INC., Boston, MA (US)

(21) Appl. No.: 18/714,813

(22) PCT Filed: Dec. 1, 2022
(86) PCT No.: PCT/US2022/051540

§ 371 Date: May 30, 2024

Related U.S. Application Data
(60) Provisional application No. 63/284,759, filed on Dec. 1, 2021.

(51) Int. Cl. A61K 35/28; A61K 35/17; A61P 35/00; C07K 16/28; C12N 5/0775

(52) U.S. Cl. CPC A61K 35/28; A61K 35/17; A61P 35/00; C07K 16/2809; C07K 16/2863; C07K 16/2866; C12N 5/0662

(57) ABSTRACT

This technology describes novel cell based combined therapeutic modalities that include a stem cell engineered to secrete a multispecific T cell engager (MiTE) polypeptide construct and an isolated population of T cells that are autologous to the subject.

### CROSS-REFERENCE TO RELATED APPLICATIONS

This application claims benefit under 35 U.S.C. § 119 (c) of U.S. Provisional Application No. 63/284,759 filed Dec. 1, 2021, the contents of which are incorporated herein by reference in their entirety.

### TECHNICAL FIELD

The field of the invention relates to cancer immunotherapy.

### BACKGROUND

Cancer is a life-threatening disease in which cells hyperproliferate, resulting in abnormal function, unregulated cell growth, lack of differentiation, local tissue invasion, and metastasis. Cancer therapies exploiting alterations in or characterized by the immune system's ability to destroy cells expressing particular cell-surface markers are showing success against cancers such as leukemias that do not form solid tumors. In particular, chimeric antigen receptor-expressing T cells (CAR-Ts) are used, which artificially target cytotoxic T cells for the killing of cancer cells. However, targeting solid tumors with this approach is proving more challenging. The solid tumor microenvironment tends to be immunosuppressive, often containing more than one layer of protection that obstructs the ability of immune cells to effectively reach and kill transformed cells of the tumor. Thus, new therapies that target solid tumors are needed for the treatment of cancer.

### SUMMARY

The methods and compositions described herein are based, at least in part, on the ability to deliver therapeutic polypeptides directly to the tumor microenvironment by exploiting the ability of stem cells, including but not limited to mesenchymal stem cells (MSCs) and neuronal stem cells (NSCs), to home to the vicinity of tumors. The compositions and methods described herein are also based, at least in part, on the discovery that combined administration of stem cells modified to express one or more therapeutic polypeptides, e.g., immunomodulatory polypeptides, with autologous T cells can potentiate the anti-cancer activity of the modified stem cells. The methods and compositions described herein are also based, at least in part, on the ability to use “off-the-shelf” or non-MHC-matched genetically-modified stem cells to deliver therapeutic polypeptides, alone or in combination with autologous, non-modified T cells to therapeutic benefit.

In one aspect, described herein is a method of treating cancer, the method comprising administering to a subject in need thereof: a) a stem cell engineered to secrete a multispecific T cell engager (MiTE) polypeptide construct; and b) an isolated population of T cells that are autologous to the subject.

In one embodiment of this or any other aspect described herein, the stem cell has a tumor tropism.

In another embodiment of this or any other aspect described herein, the stem cell is a mesenchymal stem cell (MSC) or a neuronal stem cell (NSC).

In another embodiment of this or any other aspect described herein, the stem cell is derived from an induced pluripotent stem cell.

In another embodiment of this or any other aspect described herein, the isolated population of T cells is enriched for γδ T cells.

In another embodiment of this or any other aspect described herein, the isolated population of T cells is not genetically manipulated.

In another embodiment of this or any other aspect described herein, the isolated T cell population is expanded in culture prior to administering.

In another embodiment of this or any other aspect described herein, the MiTE polypeptide comprises a binding domain that specifically binds a polypeptide expressed on the surface of a cancer cell and a binding domain that specifically binds a polypeptide expressed on the surface of a T cell.

In another embodiment of this or any other aspect described herein, the polypeptide expressed on the surface of a cancer cell is selected from the group consisting of EGFR, EGFRvIII, HER2, IL-13Rα2, EphA2 and GD2.

In another embodiment of this or any other aspect described herein, the polypeptide expressed on the surface of a T cell is selected from the group consisting of CD3, CD28, 41BB and OX40.

In another embodiment of this or any other aspect described herein, the MiTE polypeptide comprises a binding domain that specifically binds EGFR and/or EGFRvIII and a binding domain that specifically binds CD3.

In another embodiment of this or any other aspect described herein, the MiTE polypeptide comprises a binding domain that specifically binds IL13Rα2 and a binding domain that specifically binds CD3.

In another embodiment of this or any other aspect described herein, the administering comprises administering a stem cell engineered to express a first and a second MiTE polypeptide.

In another embodiment of this or any other aspect described herein, the first MiTE polypeptide comprises a binding domain that specifically binds EGFR and/or EGFRvIII, and a binding domain that specifically binds CD3, and the second MiTE polypeptide comprises a binding domain that specifically binds IL13Rα2 and a binding domain that specifically binds CD3.

In another embodiment of this or any other aspect described herein, wherein the binding domain that specifically binds CD3 specifically binds to CD38.

In another embodiment of this or any other aspect described herein, the binding domains are selected from a nanobody, a single domain antibody, and an scFv.

In another embodiment of this or any other aspect described herein, the stem cell is encapsulated in a matrix.

In another embodiment of this or any other aspect described herein, the stem cell is administered regionally, locally, or to a tumor resection cavity.

In another embodiment of this or any other aspect described herein, the stem cell is further engineered to express an immune modulator polypeptide.

In another embodiment of this or any other aspect described herein, the method further comprises administering a second stem cell population engineered to express an immune modulator polypeptide.

In another embodiment of this or any other aspect described herein, the immune modulator is selected from a cytokine and an immune checkpoint inhibitor.

In another embodiment of this or any other aspect described herein, the cytokine comprises IL12.

In another embodiment of this or any other aspect described herein, the immune checkpoint inhibitor comprises an inhibitor selected from the group consisting of PD-1, PD-L1, TIM-3, LAG-3, CTLA4, or TIGIT.

In another embodiment of this or any other aspect described herein, the stem cell is further engineered to express a polypeptide that converts a prodrug to a cytotoxic agent.

In another embodiment of this or any other aspect described herein, the method further comprises administering the prodrug to the subject.

In another embodiment of this or any other aspect described herein, the cancer is glioblastoma.

In another embodiment of this or any other aspect described herein, the T cell population is administered regionally, locally, or to a site of tumor resection.

In another embodiment of this or any other aspect described herein, the T cell population is administered intracerebroventricularly.

In another embodiment of this or any other aspect described herein, the stem cells are allogeneic to the subject.

In another embodiment of this or any other aspect described herein, the stem cells are autologous to the subject.

In another embodiment of this or any other aspect described herein, the method further comprises, before administering the stem cell, resecting a malignant tumor from the subject.

In another aspect, described herein is a composition comprising a stem cell engineered to express first and second MiTE polypeptides.

In one embodiment of this or any other aspect described herein, the first MiTE polypeptide comprises a binding domain that specifically binds a first polypeptide expressed on the surface of a cancer cell and a binding domain that specifically binds a polypeptide expressed on the surface of a T cell, and the second MiTE polypeptide comprises a binding domain that specifically binds a second polypeptide expressed on the surface of a cancer cell and a binding domain that specifically binds a polypeptide expressed on the surface of a T cell.

In another embodiment of this or any other aspect described herein, the stem cell engineered to express an immune modulator polypeptide is a mesenchymal stem cell (MSC), a neuronal stem cell (NSC) or other stem cell with a tumor tropism.

In another embodiment of this or any other aspect described herein, the engineered stem cell is formulated for delivery to a tumor resection cavity.

In another embodiment of this or any other aspect described herein, the engineered stem cell is encapsulated in a matrix.

In another embodiment of this or any other aspect described herein, the MiTE polypeptide comprises a binding domain that specifically binds EGFR or EGFRvIII, and a binding domain that specifically binds CD3, or the MiTE polypeptide comprises a binding domain that specifically binds IL13Rα2 and a binding domain that specifically binds CD3.

In another embodiment of this or any other aspect described herein, the binding domains are selected from a nanobody, a single domain antibody, and an scFv.

In another embodiment of this or any other aspect described herein, the engineered stem cell is further engineered to express an immune modulator polypeptide.

In another embodiment of this or any other aspect described herein, the immune modulator polypeptide is selected from a cytokine and an immune checkpoint inhibitor.

In another embodiment of this or any other aspect described herein, the cytokine comprises IL12.

In another embodiment of this or any other aspect described herein, the immune checkpoint inhibitor comprises an inhibitor of PD-1, PD-L1, TIM-3, LG-3, CTLA4, or TIGIT.

In another embodiment of this or any other aspect described herein, the engineered stem cell is also engineered to express a polypeptide that converts a prodrug to a cytotoxic agent.

In another embodiment of this or any other aspect described herein, the method further comprises a second stem cell population engineered to express an immune modulator polypeptide.

In another embodiment of this or any other aspect described herein, the immune modulator polypeptide expressed by the second stem cell population is selected from a cytokine and an immune checkpoint inhibitor.

In another embodiment of this or any other aspect described herein, the cytokine comprises IL12.

In another embodiment of this or any other aspect described herein, the immune checkpoint inhibitor comprises an inhibitor of PD-1, PD-L1, TIM-3, LG-3, CTLA4, or TIGIT.

In another aspect, described herein is a pharmaceutical formulation comprising an engineered stem cell as described herein. In one embodiment, the pharmaceutical composition further comprises non-engineered T cells. In one embodiment, the non-engineered T cells are autologous to a subject to whom the pharmaceutical formulation is to be administered.

In another aspect, described herein is a composition comprising a stem cell engineered to express first and second MiTE polypeptides and an isolated population of T cells. In one embodiment of this aspect or any other aspect described herein, the composition is comprised by a pharmaceutical formulation.

In another aspect, described herein is a kit comprising a composition as described herein and packaging materials therefor. In one embodiment of this aspect or any other aspect described herein, the kit comprises a genetically modified stem cell as described herein and a matrix as described herein. In another embodiment of this aspect and any other aspect described herein, the kit further comprises a prodrug. In another embodiment of this aspect and any other aspect described herein, the kit comprises a genetically modified stem cell as described herein, a matrix as described herein, a prodrug as described herein, and packaging materials therefor. In another embodiment of this aspect and any other aspect described herein, the kit further comprises a delivery device. In another embodiment of this aspect and any other aspect described herein, the kit further comprises a second stem cell engineered to express an immune modulator polypeptide.

In another aspect, described herein is a method of treating cancer, the method comprising administering to a subject in need thereof a) an immune cell engineered to secrete a multispecific T cell engager (MiTE) polypeptide construct; and b) an isolated population of T cells that are autologous to the subject.

In one embodiment of this or any other aspect described herein, the cancer is a solid tumor. In one embodiment of any aspect herein, the solid tumor is a primary tumor. In one embodiment of this or any other aspect described herein, the solid tumor is a metastatic tumor.

In one embodiment of this or any other aspect described herein, the solid tumor has been resected, or will be resected.

In one embodiment of this or any other aspect described herein, the immune cell is a macrophage or a dendritic cell.

In another aspect, described herein is a composition comprising an immune cell engineered to express first and second MiTE polypeptides.

In another aspect, described herein is a pharmaceutical formulation comprising the composition comprising an immune cell engineered to express first and second MiTE polypeptides.

### DEFINITIONS

The terms “decrease”, “reduce”, “inhibit”, or other grammatical forms thereof are used herein to mean a decrease by a statistically significant amount. In some embodiments, “reduce” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “inhibition” does not encompass a complete inhibition as compared to a reference level. “Complete inhibition” is a 100% inhibition as compared to a reference level. Where applicable, a decrease can be preferably down to a level accepted as within the range of normal for an subject without a given disease (e.g., cancer).

The terms “increased”, “increase”, “enhance”, or grammatical forms thereof are used herein to mean an increase by a statically significant amount. In some embodiments, the terms “increased”, “increase”, or “enhance”, can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., cancer. A subject can be male or female.

A subject can be one who has been previously diagnosed with or identified as suffering from or having a condition in need of treatment (e.g. glioblastoma or another type of cancer, among others) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

As used herein. “genetically modified” refers to a cell (e.g., MSC) that has been altered to introduce changes to its genetic composition. A cell can be genetically modified to contain and/or express a gene product from one or more exogenous nucleic acid sequences not found in its genome (e.g., an MSC genetically modified to express a gene product from a heterologous nucleic acid sequence). Alternatively, a cell can be genetically modified to either overexpress or inactivate or disrupt the expression of one or more genes or polypeptides. One skilled in the art will know how to introduce changes to the cell's genome using standard gene editing approaches.

In some embodiments. “activation” can refer to the state of a T cell that has been sufficiently stimulated to induce detectable cellular proliferation. T cell activation induces cytokine production including, but not limited to the production of IL-2. T cell activation can also refer to the upregulation of detectable effector functions, including but not limited to target cell cytotoxicity. At a minimum, an “activated T cell” as used herein is a proliferative T cell.

As used herein, the term “specifically binds” refers to a physical interaction between two molecules, compounds, cells and/or particles wherein the first entity binds to the second, target, entity with greater specificity and affinity than it binds to a third entity which is a non-target. In some embodiments, specific binding can refer to an affinity of the first entity for the second target, entity, which is at least 10 times, at least 50 times, at least 100 times, at least 500 times, at least 1000 times or more, greater than the affinity for the third non-target entity under the same conditions. A reagent specific for a given target is one that exhibits specific binding for that target under the conditions of the assay being utilized. A non-limiting example includes an antibody, or a ligand, which recognizes and binds with a cognate binding partner (for example, a tumor antigen or a checkpoint polypeptide) protein. For the avoidance of doubt, as used herein. “specifically binds” also requires the ability of a binding factor, such as a polypeptide or antibody binding domain to bind to a target, such as a molecule present on the cell surface, with a K_{D} of 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less. Specific binding can be influenced by, for example, the affinity and avidity of the polypeptide agent and the concentration of polypeptide agent. The person of ordinary skill in the art can determine appropriate conditions under which binding agents described herein selectively bind the targets using any suitable methods, such as titration of a polypeptide agent in a suitable cell binding assay.

In one embodiment, the term “engineered” and its grammatical equivalents as used herein can refer to one or more human-designed alterations of a nucleic acid, e.g., the nucleic acid within an organism's genome or genetic composition. The term can refer to alterations, additions, and/or deletion of genes. An “engineered cell” can refer to a cell with an added, deleted and/or altered gene. The term “cell” or “engineered cell” and their grammatical equivalents as used herein can refer to a cell of human or non-human animal origin.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

As used herein, a “checkpoint molecule” is a member of a ligand/receptor pair that exerts an inhibitory or stimulatory effect on an immune response. Immune checkpoint molecules are important in maintaining self tolerance and modulating the length and magnitude of immune responses. Tumor expression of inhibitory checkpoint molecules is a common component of tumor immune evasion and provides a target for overcoming such immune evasion to promote immune attack of the tumor. Immune checkpoint molecules can include but are not limited to PD-1 or PD-L1, CTLA4, Adenosine A2A receptor (A2AR), CD276, CD39, CD73, B7 family immune checkpoint molecules, V-set domain-containing T-cell activation inhibitor 1 (B7H4), B and T Lymphocyte Attenuator (BTLA), Indoleamine 2,3-dioxygenase (IDO), Killer-cell Immunoglobulin-like Receptor (KIR), Lymphocyte Activation Gene-3 (LAG3), nicotinamide adenine dinucleotide phosphate NADPH oxidase isoform 2 (NOX2), T-cell Immunoglobulin domain and Mucin domain 3 (TIM-3), T cell immunoreceptor with Ig and ITIM domains (TIGIT), V-domain Ig suppressor of T cell activation (VISTA), and Sialic acid-binding immunoglobulin-type lectin 7 (SIGLEC7), and those described, e.g., Pardoll et al., Nature Reviews Cancer 12, 252-264 (2012), which is incorporated herein by reference in its entirety.

As used herein, the term “checkpoint inhibitor” refers to any agent, small molecule, antibody, or the like that can reduce or inhibit the level or activity of an inhibitory immune checkpoint molecule. Inhibition of an inhibitory immune checkpoint molecule can promote an immune response, e.g., against cancer or a tumor which otherwise evades such response. Non-limiting examples of checkpoint inhibitors include pembrolizumab (Keytruda®), nivolumab (Opdivo®), cemiplimab (Libtayo®), spartalizumab, camrelizumab, sintilimab, tislelizumab, toripalimab, dostarlimab, INVMGA00012, AMP-224, AMP-514, atezolizumab (Tecentriq®), avelumab (Bavencio®), survalumab (Imfinzi®), KN035, CK-301, AUNP12, CA-170, BMS-986189, and ipilimumab (Yervoy®).

As used herein, a “prodrug” refers to a compound that can be converted from an inactive form via some chemical or physiological process in vivo (e.g., enzymatic processes and metabolic hydrolysis) to an active-form desired compound. Thus, the term “prodrug” also refers to a precursor of a biologically active compound that is pharmaceutically acceptable. A prodrug compound often offers advantages of solubility, tissue compatibility or delayed release in an organism, or provides a non-toxic or inactive agent that can be converted to a toxic or active form at a desired location (e.g., in the tumor microenvironment, or in an engineered cell) or upon the addition or removal of another agent that renders prodrug conversion to active form inducible.

The term “pharmaceutically acceptable” as used herein is consistent with the art and means compatible with the other ingredients of the pharmaceutical composition and not deleterious to the recipient thereof.

The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an agent as described herein. Each carrier must be “acceptable” in the sense of being compatible with the other ingredients of the formulation and not injurious to the patient. The terms “excipient,” “carrier,” “pharmaceutically acceptable carrier” or the like are used interchangeably herein.

In some embodiments, a nucleic acid encoding a polypeptide as described herein (e.g. an immunomodulatory polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

The term “tropism” as used herein refers to preferential movement of stem cells and/or T cells as described herein to a specific cell or tissue.

As used herein, “multispecific” refers to a molecule or construct that has specificity for at least two targets. A multispecific molecule or construct will include a separate binding domain for each of the at least two targets. A bispecific molecule or construct is a subset of multispecific molecules or constructs, which can include, for example, binding domains specific for two, three, four or more targets.

An “isolated cell” refers to a cell that is separated from other components with which it is normally associated in its natural state. For example, an isolated cell can be a cell in culture medium and/or a cell in a pharmaceutically acceptable carrier. Thus, an isolated cell can be delivered to and/or introduced into a subject. In some embodiments, an isolated cell can be a cell that is removed from a subject and manipulated as described herein ex vivo and then returned to the subject. In some embodiments, an isolated cell is an engineered cell as described herein, e.g., an engineered MSC. In some embodiments, an isolated cell is not engineered; for example, when the isolated cell is a T cell, in some embodiments the T cell is not engineered.

An “isolated T cell population” is a population of isolated cells, as that term is defined herein, which is at least 50%, 60%, 70%, 80%, 85%, 90%, 95%, 99% or more comprised of T cells. In some embodiments, an isolated T cell population is enriched for, i.e., comprises at least 50% or more, e.g., at least 50% or more, at least 60% or more, at least 70% or more, at least 80% or more, at least 85% or more, at least 90% or more, at least 95% or more, or at least 99% or more of a particular subset of T cells, e.g., γδ T cells.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. glioblastoma or other solid tumor cancer. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “administering,” refers to the placement of a therapeutic or pharmaceutical composition or agent as described herein into a subject by a method or route which results in at least partial delivery of the agent at a desired site. Pharmaceutical compositions comprising agents as described herein can be administered by any appropriate route which results in an effective treatment in the subject.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other terms are defined within the description of the various aspects and embodiments of the technology of the following.

### BRIEF DESCRIPTION OF THE DRAWINGS

FIGS. 1A-1D present data showing generation and characterization of MSC producing GBM-targeting BiTEs. (FIG. 1A) Schematic of the BiTE constructs and proteins. The engineered MSC produce the BiTE molecules which activate T cells against EGFR/EGFRvIII- and IL13Ra2-expressing GBM. (FIG. 1B) Microscope photographs in bright light and fluorescence showing different time points during co-culture of GBM cells, gel-encapsulated MSC and T cells. In the co-culture with MSC-GFP (top two rows), the tumor cells grow and there's no sign of cytotoxicity. In the co-culture with ENb-BiTE-producing MSC (bottom two rows), the accumulation of T cells above tumor cells becomes evident and leads to the disappearance of the tumor cells. (FIGS. 1C-1D) In vitro cytotoxicity assays with (FIG. 1C) ENb-BiTE-producing MSC against GBM cell lines expressing various levels of EGFR/EGFRvIII or (FIG. 1D) IL13-BiTE-producing MSC against GBM cell lines expressing various levels of IL 13Ra2. The top rows show the expression of the target antigen by flow cytometry (solid) compared to the isotype (dotted). For every cell line, the death rate is calculated respect to no-MSC controls at two different Tumor to T cell ratios (1:2 and 1:5). As expected, the cytotoxic effect is more pronounced for cells with higher expression of the target antigen. *p<0.05, ***p<0.001, ****p<0.0001, ns=not significant.

FIGS. 2A-2G present data showing BiTE-expressing MSC induce tumor regression in orthotopic models of GBM. (FIG. 2A) Schematic of the in vivo procedure. GBM cell lines were implanted orthotopically (Day 0). Two subsequent treatments after 1 and 2 weeks were performed by intra-tumoral administration of MSC producing BiTE (or GFP control) along with T cells. (FIGS. 2B-2C) bioluminescence data (left), survival curves (center), and bioluminescence images (right) of (FIG. 2B) U87 and (FIG. 2C) GBM23 models. Tumors were treated with MSC ENb-BiTE or MSC GFP as control. (FIGS. 2D-2E) bioluminescence data (left), survival curves (center), and bioluminescence images (right) of (FIG. 2D) LN229-IL13Ra2 and (FIG. 2E) GBM23 models. Tumors were treated with MSC IL 13-BiTE or MSC GFP as control. (FIGS. 2F-2G) Immuno-fluorescence staining of tissue sections from the orthotopic model reveals the presence of (FIG. 2F) CD3+ T cells in the tumor parenchyma. DAPI staining shown. A different panel reveals the presence of MSC cells in the tumor parenchyma.

FIGS. 3A-3G present data showing the combination with IL-12 improves survival in vivo. (FIG. 3A) Schematic of the GFP. IL-12 and BiTE IL-12 constructs (FIG. 3B) Schematic of the in vivo procedure. GBM cell lines were implanted orthotopically (Day 0). Two subsequent treatments after 1 and 2 weeks were performed by intra-tumoral administration of MSC producing IL-12, BiTE IL12, or GFP control along with T cells. (FIG. 3C) Western blot showing the production of the BiTEs and IL-12 from engineered MSC. (FIG. 3D-3E) bioluminescence data (left), and survival curves (right) of (FIG. 3D) U87 and (FIG. 3E) GBM23 models. Tumors were treated with MSC ENb-BiTE IL-12 or MSC GFP as control. (FIG. 3F-3G) bioluminescence data (left), and survival curves (right) of (FIG. 3F) LN229-IL13Ra2 and (FIG. 3G) GBM23 models. Tumors were treated with MSC IL13-BiTE, MSC IL-12 or MSC GFP as control.

FIGS. 4A-4G present data showing immune-profiling and effect of IL-12 in vitro. The different T cell subpopulations were analyzed after co-culture with tumor cells and MSC GFP, MSC ENb-BiTE, MSC ENb-BiTE IL-12, or no MSC. (FIG. 4A) Distribution of T helper (CD4+) and T cytotoxic cells (CD8+) (FIG. 4B) Granzyme B expression in CD4+ and CD8+ T cells (FIG. 4C) Distribution of T reg cells identified as CD4+ CD25+ CD127− FOXP3+ (FIG. 4D) Expression of FoxP3 in CD4+ CD25+ T cells (FIG. 4E) Production of IFNg and IL-2 (Th1 cytokines) or (FIG. 4F) IL-4 and IL-10 (Th2 cytokines). (FIG. 4G) Analysis of exhaustion markers PD-1, LAG-3, TIM3.

FIGS. 5A-5B present data showing twin BiTE controls tumor growth in vivo. (FIG. 5A) Schematic of the in vivo procedure. GBM cell lines were implanted orthotopically (Day 0). Two subsequent treatments after 1 and 2 weeks were performed by intra-tumoral administration of MSC TW-BiTE, or GFP control along with T cells. (FIG. 5B) bioluminescence data (left), and survival curves (right) of GBM23 model. Tumors were treated with MSC TW-BiTE or MSC GFP as control.

### DETAILED DESCRIPTION

Cancer therapies exploiting the immune system's ability to destroy cells expressing particular cell-surface markers are showing success against cancers such as leukemias that do not form solid tumors. However, targeting solid tumors is proving more challenging. The solid tumor microenvironment, it turns out, is immunosuppressive, often containing more than one layer of protection that obstructs the ability of immune cells to effectively reach and kill transformed cells of the tumor. As but one example, many tumors express immune checkpoint ligands, such as PD-L1, the normal functions of which are to assist in maintaining immune homeostasis and thereby avoid autoimmunity. A tumor expressing PD-L1 will tend to suppress the activity of cytotoxic T cells reaching the tumor by interaction with the negative immunoregulator PD-1 expressed on the T cells.

A particularly difficult scenario is presented in the case of brain tumors, such as glioblastoma, glioma and medulloblastoma. Among other things, the blood-brain barrier tends to limit access of systemically administered agents to the brain tissue and to tumors therein. Not only are glioblastomas difficult to reach with systemically administered agents, but the form in which they grow and encroach into normal brain tissues renders them difficult to remove surgically-glioblastomas, in particular, tend to extend tendrils out from the tumor mass that essentially ensure that surgery cannot capture all tumor cells.

One approach for improving tumor immunotherapy involves expression of anti-cancer immunomodulators from cells that have a natural tendency to traffic to and/or accumulate in and around tumors, i.e., cells with a tropism for tumors. In this way, the therapeutic biomolecules are delivered directly to the tumor and/or tumor microenvironment. Mesenchymal stem cells are known to home to sites of solid tumors, and can promote T cell recruitment. As such, MSCs would tend to be attractive candidates for the delivery of therapeutic molecules to the site of a tumor. However, MSCs are also well known for their immunosuppressive effects—that is, MSCs suppress T cell proliferation and cytokine production, inhibit dendritic cell expansion and function, reduce natural killer (NK) cell activity, and enhance immunosuppressive T_{reg} cell activities. See, e.g., Lee et al. Scientific Reports (2017). Therefore, while MSCs have desirable characteristics for a cell to deliver a therapeutic polypeptide, given their well-known immunosuppressive activities, one would not necessarily expect delivery of a therapeutic polypeptide to the site of a tumor by an MSC to assist with T-mediated cancer cell killing. To the contrary, one would expect the immunosuppressive activities of MSCs to limit or impede the efficacy of T cells. However, as described herein, the use of MSCs to deliver a T cell-recruiting polypeptide as described herein can potentiate T cell-mediated cancer cell killing. In addition to MSCs, neuronal stem cells (NSCs) have also been demonstrated to have a natural tropism for tumors, including neuronal and brain tumors. Thus, one component of the cancer therapeutic approaches described herein is the use of engineered stem cells, including MSCs, NSCs or other tumor-trophic stem cells, to deliver an anti-tumor payload or payloads to a tumor or tumor microenvironment.

In one embodiment, the stem cells are engineered to express a recombinant protein construct that targets T cells to a tumor or tumor microenvironment. An important class of such constructs are so-called “T cell engager” polypeptides. These multispecific constructs, which are described further below, include at least a binding domain that specifically binds to a protein expressed on the surface of a T cell, as that term is described and defined herein, and a binding domain that specifically binds to a protein expressed on the surface of a tumor cell, e.g., a tumor antigen as that term is described and defined herein. When expressed as a fusion protein including the two binding domains, generally connected by a linker domain, the resulting construct can bind to a tumor cell and facilitate the recruitment and binding of a T cell in the tumor's microenvironment, thus allowing the T cell to effectively target the tumor's cells. Where such constructs have two different specific binding domains, they have become known as “Bispecific T-cell Engagers,” or “BiTEs.” Such constructs, which in some embodiments can include further binding domains, are also referred to herein as “Multispecific T-cell Engagers,” or “MiTEs.” For the avoidance of doubt, BiTEs are a subset of MiTEs, for example, a BiTE is a MiTE that includes two binding domains.

In one embodiment, a stem cell engineered to express and secrete an immunotherapeutic polypeptide, including but not limited to a MiTE, can be administered in combination with autologous T cells. Providing an increased supply of autologous T cells to interact with the MiTE expressed by the stem cell can further potentiate the engagement of T cells to attack the tumor. Such T cells can be isolated from the subject prior to use in combination with an engineered stem cell, and can be, for example, expanded in culture to increase cell number. While the autologous T cells administered in conjunction with engineered stem cells as described herein need not be engineered, it can be beneficial to treat or manipulate isolated T cells to enrich for or promote expansion of one or more sub-populations, such as γδ T cells.

The resection of a tumor promotes a reduction of myeloid-derived suppressor cells and a simultaneous recruitment of CD4/CD8 T cells. As such, apart from simply removing the cancerous tissue, resection can assist in countering the immunosuppressive tumor microenvironment, and can be combined with the approach of administering stem cells (e.g., MSC) expressing a MiTE polypeptide and a T cell population as described herein, to beneficial effect.

Accordingly, one aspect of the technology described herein provides a method for treating a solid tumor comprising administering a) a stem cell engineered to secrete a MiTE polypeptide construct; and b) an isolated population of T cells that are autologous to the subject.

In one embodiment, a subject is administered at least one stem cell population engineered to express a MiTE and autologous T cells. In another embodiment, a subject is administered at least one stem cell population engineered to express a MiTE and another therapeutic agent, e.g., a cytokine, checkpoint inhibitor, or other agent or immunomodulator. Where the stem cell is engineered to express more than one type of therapeutic agents, the more then one type of agent can be the same (e.g., two different MiTES) or different (e.g., a MiTE and a cytokine, or a MiTE and a checkpoint inhibitor).

In another embodiment, a subject is administered at least two stem cell populations, wherein the stem cell populations are engineered to express at least one therapeutic agent (i.e., at least 1, 2, 3, or more therapeutic agents), e.g., a MiTE, cytokine, checkpoint inhibitor or other agent or immunomodulator. Where more than one stem cell population is administered, the more than one population can be engineered to express the same or different types of therapeutic agents (e.g., two stem cell populations engineered to express MiTES) or different (e.g., a stem cell population engineered to express a MiTE and a stem cell population engineered to express a cytokine, checkpoint inhibitor or other agent or immunomodulator).

Another aspect of the technology described herein provides a method for treating a solid tumor comprising administering a) an immune cell engineered to secrete a MiTE polypeptide construct; and b) an isolated population of T cells that are autologous to the subject.

In one embodiment, the immune cell is a macrophage or a dendritic cell.

In one embodiment, the immune cell is a peripheral monocyte, e.g., from a patient. In one embodiment, the peripheral monocyte can be modified to express a MiTE, such that the MiTE is expressed by a dendritic cell differentiated from the monocyte.

Another aspect of the technology described herein is a composition comprising a stem cell engineered to express at least one MiTE polypeptide.

Another aspect of the technology described herein is a composition comprising a stem cell engineered to express first and second MiTE polypeptides.

Another aspect of the technology described herein is a composition comprising an immune cell engineered to express at least one MiTE polypeptide.

Another aspect of the technology described herein is a composition comprising an immune cell engineered to express first and second MiTE polypeptides.

### MiTEs

In certain embodiments, a MiTE polypeptide described herein comprises at least one binding domain that specifically binds a polypeptide expressed on the surface of a cancer cell, for example, a tumor antigen, and at least one binding domain that specifically binds a polypeptide expressed on the surface of a T cell. The following describes the various components of a MiTE as described herein.

### Tumor Antigens

Where a MiTE includes a binding domain that specifically binds a tumor antigen, the term “tumor antigen” refers to antigens which are differentially expressed by cancer cells and can thereby be exploited in order to target cancer cells. Tumor antigens are antigens which can potentially stimulate tumor-specific immune responses. Some of these antigens are encoded, although not necessarily expressed, by normal cells. A non-limiting example of such an antigen is the epidermal growth factor receptor, EGFR, which can be expressed in normal tissues, but when overexpressed or activated, e.g., by mutation, can act as an oncogene. These antigens can be characterized as those which are normally silent (i.e., not expressed) or closely regulated in normal cells, those that are expressed only at certain stages of differentiation and those that are temporally expressed such as embryonic and fetal antigens. Other tumor antigens are encoded by mutant cellular genes, such as oncogenes (e.g., activated ras oncogene), suppressor genes (e.g., mutant p53), and fusion proteins resulting from internal deletions or chromosomal translocations. Still other tumor antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), HER2, mucins (i.e., MUC-1), prostate-specific antigen (PSA), prostatic acid phosphatase (PAP) IL-13Rα2, EphA2 and GD2. In addition, viral proteins such as some encoded by hepatitis B (HBV), Epstein-Barr (EBV), and human papilloma (HPV) have been shown to be important in the development of hepatocellular carcinoma, lymphoma, and cervical cancer, respectively. The term “polypeptide expressed on the surface of a cancer cell” is synonymous and used interchangeably with “tumor antigen” as described herein. Table 2 describes non-limiting examples of tumor antigens associated, for example, with several representative types of cancer.

TABLE 2
Tumor antigens associated with a given cancer.

| Cancer | Tumor Antigen |
|---|---|
| Glioblastoma | EGFRvIII |
| Glioma | HER2 |
| Medulloblastoma | CD133 |
| | EGFR |
| | IL13RA2 |
| Melanoma | L1-CAM |
| | CTAG1B |
| | GD2 |
| Breast Cancer | HER2 |
| Non-small Cell Lung Cancer | EGFR |

In one embodiment, the tumor antigen is EGFR or a variant thereof, such as EGFRvIII. EGFR is a transmembrane protein that is a receptor for members of the epidermal growth factor family of extracellular protein ligands. EGFR is a member of the ErbB family of receptors. EGFR sequences are known for a number of species, e.g., human EGFR (NCBI Gene ID: 1956), mRNA (NCBI Ref Seq NM_001346897.1), and polypeptide sequences (NP_001333826.1, SEQ ID NO: 26), and mouse polypeptide sequences (NP_997538.1, SEQ ID NO: 27). EGFR can refer to human EGFR, including naturally occurring variants and alleles thereof. In some embodiments of, e.g., in veterinary applications, EGFR can refer to the EGF receptor of, e.g., dog, cat, cow, horse, pig, and the like. Homologs and/or orthologues of human EGFR are readily identified for such species by one of skill in the art, e.g., using the NCBI orthologues search function or searching available sequence data for a given species for sequence similar to a reference EGFR sequence.

Over expression of EGFR and mutations resulting in EGFR variants have been linked to disease states, including cancer. Specifically, aberrant signaling of EGFR variant III (EGFRvIII) has been shown to be important in driving tumor progression and often correlates with poor prognosis; EGFRvIII is associated with increased proliferation of glioma cells in subjects diagnosed with glioblastoma.

EGFRvIII is the most common extracellular mutation of EGFR, and is also known as de2-7EGFR and ΔEGFR. EGFRvIII results from in-frame deletion of 801 base pairs spanning exons 2-7 of the EGFR coding sequence, resulting in the deletion of 267 amino acids from the extracellular domain. Examples of antibodies that bind to EGFRvIII can be found, e.g., in US Patent Application No. US20140322275A1, which is incorporated herein by reference, as summarized in Table 1.

### Polypeptides Expressed on the Surface of a T Cell

Where a MiTE as described herein includes a binding domain that specifically binds a polypeptide expressed on the surface of a T cell, the term “polypeptide expressed on the surface of a T cell” refers to a polypeptide that is characteristic of a T cell. A goal of the multispecific T cell engagers described herein is to use a construct that binds a tumor cell antigen and a T cell marker so as to facilitate bringing the T cell into very close proximity with the tumor cell. To achieve this goal without also binding the constructs to non-T cell cells (which would dilute the construct's effectiveness and potentially lead to side effects), the T cell marker or polypeptide expressed on the surface of a T cell bound by the multispecific T cell engager is ideally one that is only expressed on T cells. Non-limiting examples of such polypeptides expressed on the surface of a T cell include CD3 (including CD3 epsilon, gamma, delta and zeta), CD113, and CD161 among others. Some T-cell-specific markers are only expressed on subsets of T cells; non-limiting examples include CD127, which is expressed on naïve and memory CD4+ and CD8+ T cells, and CD37 and CD152, which are expressed on activated T cells.

### Binding Domains

As discussed above, a MiTE as described herein will include at least two binding domains—at least one that specifically binds a polypeptide expressed on the surface of a cancer cell, e.g., a tumor antigen, and at least one that specifically binds a polypeptide expressed on the surface of a T cell. While other protein-binding domains that permit specific, high affinity binding to a tumor antigen or T cell polypeptide are contemplated, one class of binding domain suited for use in a MiTE as described herein is an antigen-binding domain of an antibody.

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions or antigen-binding fragments thereof. An antibody agent can include, but is not limited to chimeric antibody, a CDR-grafted antibody, a humanized antibody, a multispecific antibody, a dual specific or bispecific antibody, an anti-idiotypic antibody, a functionally active epitope-binding portion thereof, and/or a bifunctional hybrid antibody. In preferred embodiments, the binding domains of a MiTE as described herein will comprise scFvs, nanobodies, or a combination thereof.

The terms “antigen-binding fragment” and “antigen-binding domain” are used herein to refer to one or more fragments of a full length antibody that retain the ability to specifically bind to a target antigen of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)₂ fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546), which consists of a VH or VL domain; and (vi) an isolated complementarity determining region (CDR) that retains specific antigen-binding functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883.

In some embodiments, the antibody agent is a nanobody. As used herein, a “nanobody” refers to a single-domain antibody comprising a single monomeric variable antibody domain. A nanobody selectively binds to a specific antigen, similar to an antibody. A nanobody is typically small in size, ranging from 12-15 kDa. Methods for designing and producing nanobodies are known in the art and are further described in Ghahroudi, et al. FEBS Letters. September 1997, 414:3 (521-526), which is incorporated herein in its entirety by reference. See also PCT/EP93/02214 (WO 94/04678 published 3 Mar. 1994), which is incorporated by reference herein in its entirety).

In some embodiments, the antibody or antigen-binding portion thereof is a fully human antibody. In some embodiments, the antibody, antigen-binding portion thereof, is a humanized antibody or antibody reagent. In some embodiments, the antibody, antigen-binding portion thereof, is a fully humanized antibody or antibody reagent. In some embodiments, the antibody or antigen-binding portion thereof, is a chimeric antibody or antibody reagent. In some embodiments, the antibody, antigen-binding portion thereof, is a recombinant polypeptide. In some embodiments, the chimeric T cell antigen receptor comprises an extracellular domain that binds EGFRvIII or other tumor antigen wherein the extracellular domain comprises a humanized or chimeric antibody or antigen-binding portion thereof.

The term “human antibody” refers to antibodies whose variable and constant regions correspond to or are derived from immunoglobulin sequences of the human germ line, as described, for example, by Kabat et al. (see Kabat, et al. (1991) Sequences of Proteins of Immunological Interest, Fifth Edition, U.S. Department of Health and Human Services, NIH Publication No. 91-3242). However, the human antibodies can contain amino acid residues not encoded by human germ line immunoglobulin sequences (for example mutations which have been introduced by random or site-specific mutagenesis in vitro or by somatic mutation in vivo), for example in the CDRs, and in particular in CDR3. Recombinant human antibodies as described herein have variable regions and may also contain constant regions derived from immunoglobulin sequences of the human germ line (see Kabat, E. A., et al. (1991) Sequences of Proteins of Immunological Interest. Fifth Edition, U.S. Department of Health and Human Services. NIH Publication No. 91-3242). According to particular embodiments, however, such recombinant human antibodies are subjected to in-vitro mutagenesis (or to a somatic in-vivo mutagenesis, if an animal is used which is transgenic due to human Ig sequences) so that the amino acid sequences of the VH and VL regions of the recombinant antibodies are sequences which although related to or derived from VH and VL sequences of the human germ line, do not naturally exist in vivo within the human antibody germ line repertoire. According to particular embodiments, recombinant antibodies of this kind are the result of selective mutagenesis or back mutation or of both. Preferably, mutagenesis leads to an affinity to the target which is greater, and/or an affinity to non-target structures which is smaller than that of the parent antibody. Generating a humanized antibody from the sequences and information provided herein can be practiced by those of ordinary skill in the art without undue experimentation. In one approach, there are four general steps employed to humanize a monoclonal antibody, see, e.g., U.S. Pat. Nos. 5,585,089; 6,835,823; 6,824,989. These are: (1) determining the nucleotide and predicted amino acid sequence of the starting antibody light and heavy variable domains; (2) designing the humanized antibody, i.e., deciding which antibody framework region to use during the humanizing process; (3) the actual humanizing methodologies/techniques; and (4) the transfection and expression of the humanized antibody.

Usually the CDR regions in humanized antibodies and human antibody variants are substantially identical, and more usually, identical to the corresponding CDR regions in the mouse or human antibody from which they were derived. In some embodiments, it is possible to make one or more conservative amino acid substitutions of CDR residues without appreciably affecting the binding affinity of the resulting humanized immunoglobulin or human antibody variant. In some embodiments, substitutions of CDR regions can enhance binding affinity.

In some embodiments, the checkpoint inhibitor polypeptide as described herein comprises an antibody or antigen-binding domain thereof that binds a checkpoint polypeptide selected from the group consisting of PD-L1, PD-1, CTLA-4, TIM-3, LAG-3, or TIGIT. In some embodiments, the antibody binds to an amino acid sequence complimentary to any one of the sequences described herein SEQ ID NO: 1-27. Table 1 summarizes examples of antibodies and their binding domains that can be used in the compositions and methods described herein.

TABLE 1
SUMMARY OF ANTIBODY TARGETS AND ANTIBODIES

| | Protein | | | |
|---|---|---|---|---|
| Polypeptide | SEQ ID NOs: | Antibodies | Antibody SEQ ID NOs: | References |
| CTLA-4 | SEQ ID | ipilimumab (CTLA-4; | SEQ ID NO: 16, | Metzler et al. Nat |
| | NO: 3, | Bristol Meyers Squibb); | SEQ ID NO: 17 | Struct Biol. 1997; |
| | SEQ ID | tremelimumab (CTLA-4; | | US20150283234 |
| | NO: 8 | Medimmune); | | |
| PD-1 | SEQ ID | pembrolizumab (PD-1; | SEQ ID NO: 18, | US2012135408; |
| | NO: 2, | Merck); nivolumab (PD-1; | SEQ ID NO: 19, | US2013173223 |
| | SEQ ID | Bristol Meyers Squibb); | SEQ ID NO: 20, | |
| | NO: 15 | AUNP12 (PD-1; Aurigene); | SEQ ID NO: 21 | |
| PD-L1 | SEQ ID | atezolizumab (PD-L1; | SEQ ID NO: 22 | US8217149B2 |
| | NO: 1, | Genentech); MPDL3280A | SEQ ID NO: 23 | |
| | SEQ ID | (PD-L1; Roche); | | |
| | NOs: 12-14 | MEDI4736 (PD-L1; | | |
| | | AstraZeneca); | | |
| | | MSB0010718C (PD-L1; | | |
| | | EMD Serono); avelumab | | |
| | | (PD-L1; Merck); | | |
| | | MSB0010718C (PD-L1; | | |
| | | EMD Serono); durvalumab | | |
| | | (PD-L1; Medimmune); | | |
| TIM-3 | SEQ ID | TSR-022 (TIM3; Tesaro) | SEQ ID NO: 24; | WO2018129553A1 |
| | NO: 4, | | SEQ ID NO: 25 | |
| | SEQ ID | | | |
| | NO: 10 | | | |
| LAG-3 | SEQ ID | IMP321 (LAG3: | See Sequences in | US9579382B2; |
| | NO: 5, | Immuntep); BMS-986016 | US20110150892A1 | US20110150892A1 |
| | SEQ ID | (LAG3; Bristol Meyers | | |
| | NO: 11 | Squibb); | | |
| TIGIT | SEQ ID | Anti-Tigit (Compugen Ltd) | See Sequences (e.g. | US20180280506A1 |
| | NOs: 6-7 | | SEQ ID NOs: 160 | |
| | | | and 165) in | |
| | | | US20180280506A1 | |
| EGFRvIII | SEQ ID | Anti-EGFRvIII (University | See Sequences (e.g. | US20140322275A1 |
| | | of Pennsylvania, Novartis | SEQ ID NO: 11) in | |
| | NOs: 26-27 | Inst for BioMedical Res | US20140322275A1 | |
| | | Inc.) | | |

### Stem Cells

A stem cell is a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors.

In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Generally, “progenitor cells” have a cellular phenotype that is more primitive (i.e., is at an earlier step along a developmental pathway or progression than is a fully differentiated cell). Often, progenitor cells also have significant or very high proliferative potential. Progenitor cells can give rise to multiple distinct differentiated cell types or to a single differentiated cell type, depending on the developmental pathway and on the environment in which the cells develop and differentiate.

In the context of cell ontogeny, the adjective “differentiated”, or “differentiating” is a relative term. A “differentiated cell” is a cell that has progressed further down the developmental pathway than the cell it is being compared with. Thus, stem cells can differentiate to lineage-restricted precursor cells, which in turn can differentiate into other types of precursor cells further down the pathway, and then to an end-stage differentiated cell, which plays a characteristic role in a certain tissue type, and may or may not retain the capacity to proliferate further.

### Mesenchymal Stem Cells

In one embodiment, the stem cell as described herein is a mesenchymal stem cell (MSC). A mesenchymal stem cell (MSC) is a self-renewing, multipotent stem cell that comprises the capacity to differentiate into various cell types including, but not limited to, white adipocytes, brown adipocytes, myoblasts, skeletal muscle, cardiac muscle, smooth muscle. chondrocytes, and mature osteoblasts upon introduction of proper differentiation cues. An MSC can be produced using techniques known in the art, for example, by a process comprising obtaining a cell by dispersing an embryonic stem (ES) cell colony and culturing the cell with MSC conditioned media. MSCs also occur in and can be prepared from bone marrow. Methods of isolating, purifying and expanding mesenchymal stem cells (MSCs) are known in the art and include, for example, in U.S. Pat. No. 5,486,359 and Jones E. A. et al., 2002, Isolation and characterization of bone marrow multipotential mesenchymal progenitor cells, Arthritis Rheum. 46(12): 3349-60. A method of isolating mesenchymal stem cells from peripheral blood is described by Kassis et al. [Bone Marrow Transplant. 2006 May; 37(10);967-76]. A method of isolating mesenchymal stem cells from placental tissue is described by Zhang et al. [Chinese Medical Journal, 2004, 117 (6):882-887]. Methods of isolating and culturing adipose tissue, placental and cord blood mesenchymal stem cells are described by Kern et al. [Stem Cells, 2006; 24:1294-1301]. A method of isolating mesenchymal stem cells from umbilical cord blood; is described by Lee et al., Isolation of multipotent mesenchymal stem cells from umbilical cord blood, Blood 103: 1669-1675 (2004). A population of MSCs can be confirmed by assessing cell surface markers. For example, at a minimum, 95% or more of an MSC cell population expresses CD73/5′-Nucleotidase, CD90/Thy1, and CD105/Endoglin. MSCs do not express committed-lineage specific markers such as CD3, CD7, CD14, CD19, CD38, CD66b, or glycophorin. The expression of these surface markers can be assessed using techniques known in the art, e.g., FACS analysis. Cell surface and/or intracellular markers can also be assessed, for example, via RT-PCR. While not wishing to be ound by theory, the tumor tropism of MSCs (movement or migration to, or accumulation at the site of a tumor) is thought to be driven by paracrine signaling between the tumor microenvironment and the corresponding receptors on the cell surface of the MSC.

Importantly, mesenchymal stem cells have been found to have both immunoenhancing as well as immunosuppressive properties; they have an effect on innate and specific immune cells. Mesenchymal stem cells have been reported to produce many immunomodulatory molecules including prostaglandin E2 (PGE2), [21] nitric oxide, [22] indoleamine 2,3-dioxygenase (IDO), interleukin 6 (IL-6), and other surface markers such as FasL, [23] PD-L1 and PD-L2, among others.

MSCs recruit monocytes, T cells and dendritic cells to sites of inflammation following an infection or injury (e.g., tumor resection) via expression of chemokine (C-C motif) ligand 2 (CCL2, as known as MCP-1 and small inducible cytokine A2). It is contemplated that an MSC genetically modified to express increased levels of CCL2 (compared to wild-type CCL2 levels) will have a greater capacity to recruit T cells to a site of injury (e.g., tumor resection) compared to a wild-type MSC. CCL2 sequences are known for a number of species, e.g., human CD28 (NCBI Gene ID: 6347). CCL2 can refer to human CCL2, including naturally occurring variants and alleles thereof. In some embodiments of, e.g., in veterinary applications. CCL2 can refer to the CCL2 of, e.g., dog, cat, cow, horse, pig, and the like. Homologs and/or orthologs of human CCL2 are readily identified for such species by one of skill in the art, e.g., using the NCBI ortholog search function or searching available sequence data for a given species for sequence similar to a reference CCL2 sequence.

As noted above, MSCs are recognized for their immunosuppressive activity. Without wishing to be bound by theory, this immunosuppression is believed to be mediated in several different ways, including suppression of NK cell proliferation and effector function, suppression of dendritic cell development and maturation, and induction of T regulatory (T_{reg}) expansion, among others. These properties likely play a role in the immune privileged, or immune-evasive, characteristics of MSCs that permit non-MHC-matched allotransplantation without provoking a robust rejection response. While allgeneic MSC transplants do not necessarily completely avoid rejection in the long term, their unique immune status or low immune profile, coupled with their ability to deliver therapeutic agents to the tumor microenvironment makes them well suited as “off-the-shelf” therapeutic delivery agents. By this is meant that MSCs can be genetically modified as described herein to express, for example, a MiTE and/or other therapeutic or immunomodulatory polypeptide agent(s), expanded and stored, e.g., frozen. The modified, expanded and stored MSCs can then be used for administration to non-MHC-matched recipients. It is noted that even if transplanted non-autologous or non-MHC matched cells eventually succumb to the recipient's immune attack, they are not needed after a cancer has successfully been treated by the therapeutic agent they deliver.

It is specifically contemplated that different populations of MSCs can be modified to express different therapeutic or immunomodulatory agents, e.g., expressing MiTEs targeting different tumor antigens (or different cell surface markers expressed by T cells), or expressing different checkpoint inhibitors or modulators, and/or expressing different cytokines can be prepared, expanded and stored to permit off-the-shelf allogeneic transplant, e.g., when a subject has a tumor resected, whether administered systemically or at the site of resection. Because the allogeneic MSC transplant need not be MHC matched, there are benefits in scale, time and cost, because one need not separately prepare genetically-modified autologous MSCs for each patient.

Thus, in one embodiment, the stem cell used herein is an “off-the-shelf” genetically modified mesenchymal stem cell for therapeutic use. It is specifically contemplated herein that the use of off-the-shelf genetically modified MSCs will advantageously shorten the production time for the technology described herein, and create a more cost-efficient way for the same.

### Induced Pluripotent Stem Cells

In some embodiments, the stem cells described herein are derived from isolated pluripotent stem cells (iPSCs). Although differentiation is generally irreversible under physiological contexts, several methods have been developed to reprogram somatic cells to induced pluripotent stem cells. Exemplary methods are known to those of skill in the art and are described briefly herein below.

As discussed above, MSCs do not necessarily need to be MHC matched between donor and transplant recipient to provide therapeutic benefit. However, an advantage of using iPSCs is that the cells can be derived from the same subject to which the progenitor cells are to be administered. That is, a somatic cell can be obtained from a subject, reprogrammed to an induced pluripotent stem cell, and then re-differentiated into desired differentiated cell to be administered to the subject (i.e., autologous cells). Since the progenitor cells are essentially derived from an autologous source, the risk of engraftment rejection or allergic responses is reduced compared to the use of cells from another subject or group of subjects. In some embodiments, the progenitor cells are derived from non-autologous sources. In addition, the use of iPSCs negates the need for cells obtained from an embryonic source. Thus, in one embodiment, the stem cells used in the disclosed methods are not embryonic stem cells, iPS cells can be differentiated to an MSC or NSC phenotype.

The process of generating iPS cells from somatic cells is referred to as “reprogramming,” and essentially drives the differentiation of a cell backwards to a more undifferentiated or more primitive type of cell.

The cell to be reprogrammed can be either partially or terminally differentiated prior to reprogramming. In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell).

The specific reprogramming approach or method used to generate pluripotent stem cells from somatic cells is not critical to the claimed technology. Thus, any method that re-programs a somatic cell to the pluripotent phenotype would be appropriate for use in the methods described herein.

Reprogramming methodologies for generating pluripotent cells using defined combinations of transcription factors have been described. Yamanaka and Takahashi converted mouse somatic cells to ES cell-like cells with expanded developmental potential by the direct transduction of Oct4, Sox2, Klf4, and c-Myc (Takahashi and Yamanaka, 2006).

Subsequent studies have shown that human iPS cells can be obtained using similar transduction methods (Lowry et al., 2008; Park et al., 2008; Takahashi et al. 2007; Yu et al., 2007b), and the transcription factor trio, OCT4, SOX2, and NANOG, has been established as the core set of transcription factors that govern pluripotency (Jaenisch and Young, 2008). The production of iPS cells can be achieved by the introduction of nucleic acid sequences encoding stem cell-associated genes into an adult, somatic cell, historically using viral vectors.

iPS cells can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., Cell Stem Cell, 2010 Nov. 5;7(5):618-30). Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. In one embodiment, reprogramming can comprise introducing one or more of Oct-3/4, a member of the Sox family, a member of the Klf family, and a member of the Myc family to a somatic cell. In one embodiment, the methods and compositions described herein further comprise introducing one or more of each of October 4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one embodiment the reprogramming is not effected by a method that alters the genome. Thus, in such embodiments, reprogramming is achieved, e.g., without the use of viral or plasmid vectors.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135

To confirm the induction of pluripotent stem cells for use in generating MSCs (or T cells) useful in the methods and compositions described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

iPS cells can be differentiated to MSCs using methods known in the art. As one example, see Yang et al., Cell Death & Disease 10: 718 (2019), iPSCs can also be differentiated to neuronal or neural stem cells e.g., as described by Jendelova et al., Results Probl. Cell Differ. 66: 89-102 (2018).

### Genetically Modified Stem Cells

In certain embodiments, a stem cell (e.g., MSC, NSC, etc.) as described herein is genetically modified to express at least one MiTE polypeptide construct. Alternatively, a stem cell as described herein can be genetically engineered to express a first and a second MiTE polypeptide. It is contemplated herein that such first and the second MiTE polypeptides differ in at least one binding domain. For example, the first and the second MiTE polypeptides can comprise the same binding domains that specifically bind a polypeptide expressed on the surface of a cancer cell, and comprise different binding domains that specifically bind a polypeptide expressed on the surface of a T cell, or vice versa.

In one embodiment, the stem cell is further engineered to express at least one immune modulator.

MiTEs, immune modulators or other exogenously-introduced factors can be introduced to a cell using a vector carrying nucleic acid sequence encoding the factor(s). Various vectors and systems for introducing exogenous constructs to cells are known in the art, and include plasmid vectors, viral vectors, cosmids and the like.

A vector is preferably an expression vector that drives the expression of the transgene or construct in the target cell. Expression can be rendered constitutive, tissue-specific, or inducible if so desired by inclusion of appropriate regulatory sequences on the vector.

Integrating vectors have their delivered nucleic acid permanently incorporated into the host cell chromosomes. Non-integrating vectors remain episomal, such that the nucleic acid contained therein is not integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Other non-integrative viral vectors include adenoviral vectors and the adeno-associated viral (AAV) vectors. Adenoviral vectors and their use to introduce genetic material to cells are reviewed, for example, by Douglas, J. T., Molecular Biotechnol. 36: 71-80 (2007), Palmer & Ng, Human Gene Ther. 16: (February 2005) and Volpers & Kochanek, J. Gene Med. 6: Supp. 1: S164-171 (2004).

### Prodrugs

In certain embodiments, it can be advantageous to design a kill switch into genetically modified cells, such as genetically modified stem cells as described herein. For example, a kill switch can be useful to selectively kill the introduced cells after they have effected tumor cell killing, for example, to avoid or limit any immune response or other unwanted activity that might occur with longer residence of the exogenous cells. One way to achieve such selective killing is to include a heterologous gene in the introduced cells that renders the cells sensitive to a drug that will selectively kill those cells. In one embodiment the kill switch or inducible cell removal system converts a prodrug from a non-toxic form to a toxic form. If the genetically modified stem cells described herein for tumor treatment carry such a gene, once anti-tumor treatment is judged to be complete or adequately effective, one can administer the prodrug, which will only be converted to the toxic form by the gene product in the introduced cells, thereby selectively killing the introduced cells while substantially sparing the subject host's own cells. Examples of gene/prodrug systems are known in the art. As but one non-limiting example, non-toxic ganciclovir can be converted to toxic ganciclovir triphosphate by HSV thymidine kinase (HSV-TK). The 2′-deoxyguanosine analogue ganciclovir is not efficiently metabolized to its active DNA synthesis-inhibiting form in cells lacking certain viral thymidine kinase (TK) enzymes (e.g., HSV-TK, CMV-TK), but in cells expressing such thymidine kinase enzymes, the ganciclovir pro-drug is efficiently metabolized to ganciclovir triphosphate, which is a competitive inhibitor of dGTP incorporation into DNA, leading to cell death. Thus, inclusion of the HSV-TK coding sequence on a construct introduced when modifying stem cells as described herein can provide a kill switch to selectively eradicate the introduced stem cells once they are no longer desired or needed. Constructs encoding the HSV-TK enzyme and their coupling with the ganciclovir prodrug system are described, for example, by Chiu et al. Int. J. Cancer 102: 328-333 (2002), and Kim et al., Cancer Gene ther. 7: 240-246 (2000) and references cited therein. Other pro-drugs and agents or enzymes that promote their conversion to active form are known in the art. Non-limiting, exemplary prodrug converting enzymes with their prodrug partners include, but are not limited to, herpes simplex virus thymidine kinase/gancyclovir, varicella zoster thymidine kinase/gancyclovir, cytosine deaminase/5-fluorouracil, purine nucleoside phosphory lase/6-methylpurine deoxyriboside, beta lactamase/cephalosporin-doxorubicin, carboxypeptidase G2/4-[(2-chloroethyl)(2-mesuloxyethyl)amino]benzoyl-L-glutamic acid, cytochrome P450/acetominophen, horseradish peroxidase/indole-3-acetic acid, nitroreductase/CB 1954, rabbit carboxylesterase/7-ethyl-10-[4-(1-piperidino)-1-piperidino]carbonyloxycam-potothecin, mushroom tyrosinase/bis-(2-chloroethyl)amino-4-hydroxyphenylaminomethanone 28, beta galactosidase/1-chloromethyl-5-hydroxy-1,2-dihyro-3H-benz[e]indole, beta glucuronidase/epirubicin-glucoronide, thymidine phosphorylase/5′-deoxy-5-fluorouridine, deoxycytidine kinase/cytosine arabinoside, beta-lactamase and linamerase/linamarin. Coding sequences for the various prodrug converting enzymes are known in the art.

Such pro-drug systems can provide a heterologous inducible cell suicide system. As used herein, a “heterologous inducible cell suicide system” is a system for selectively killing engineered stem cells as described herein. Such systems involve the introduction of one or more heterologous nucleic acid sequences to the stem cell that render the cell responsive to a cell death-inducing agent. The system is maintained in an inactive state until the inducing agent, e.g., a small molecule or other drug, is administered to the patient. Different configurations of heterologous inducible cell suicide systems include, but are not limited to one in which the cell is modified to express an enzyme that converts a non-toxic pro-drug to a toxic form, and one in which the cell is modified to contain a nucleic acid construct encoding a cell death inducing polypeptide under control of a genetic element inducible by a small molecule or other drug. Various embodiments of such systems are known in the art and/or described further herein. As used herein, the term “inducible” refers to a system that is substantially inactive until an inducing agent is provided. The term can refer, for example, to a gene or genetic element the expression of which is inducible by addition of a drug, such as a tetracycline- or doxycycline-inducible construct, or to a heterologous cell suicide system in which cell suicide is induced by the addition of a drug. By “substantially inactive” in the context of a heterologous inducible cell suicide system is meant that in the absence of the inducing drug, the inducible system maintains expression of the cell killing machinery at a level that permits the cell to remain viable, home to a tumor, and produce one or more therapeutic agents or polypeptides.

In one embodiment, a MiTE described herein is a T cell engager prodrug designed to be conditionally active in a tumor microenvironment. In some cases, this enables targeting of a wider selection of tumor antigens (e.g., solid tumor antigens). In some embodiments, a MiTE combines the desirable attributes of several prodrug approaches, including, but not limited to: combination of steric and specific masking; additional safety imparted by half-life differential of prodrug versus an active drug, derived by activation of the conditionally activated MiTE; and ability to plug-and-play with different tumor target binders.

### Immune Modulators

In one embodiment, a stem cell (e.g., MSC, NSC, etc.) is genetically modified to express a polypeptide comprising an immune modulator. In one embodiment, a stem cell (e.g., MSC, NSC, etc.) that expresses a MiTE polypeptide is further genetically modified to express a polypeptide comprising an immune modulator. An “immune modulator,” or alternatively an “immunomodulator” refers to an agent with the capacity to modify the immune system or function thereof in a subject. For example, an immunmodulator can induce, amplify, attenuate, or prevent an immune response. It should be understood that while any antigen can be an immunomodulator in the sense that it induces an immune response, an “immune modulator” as the term is used herein modifies the immune microenvironment of a tumor, e.g., in terms of recruitment of immune effectors, activity of immune effectors, or suppression of immunosuppressive factors, their expression, or their activity. In some embodiments, an immune modulator is a polypeptide that modulates the activity of an immune-related pathway or process, and includes, for example, cytokines, immune checkpoint inhibitors or modulators, Immune modulators useful in the methods and compositions described herein promote or assist in the promotion of an anti-tumor immune response; thus, an immune modulator either directly promotes or increases an anti-tumor immune response or inhibits an immunosuppressive function to thereby promote an anti-tumor immune response.

In one embodiment, the immune modulator expressed by a genetically modified stem cell potentiates cancer cell killing. That is, the immune modulator increases cancer cell killing, e.g., by T cells. The increase can be relative, for example, to the killing mediated by T cells alone, or, alternatively, killing mediated by genetically modified stem cells as described herein that express a MiTE.

Immune modulating cytokines include, for example, chemokines, inteferons, interleukins, lymphokines and tumor necrosis factors. Cytokines that promote anti-cancer responses are described, for example, by Berraondo et al., Brit. J. Cancer 120: 6-15 (2019).

Immune checkpoint molecules include, but are not limited to PD-1, PD-L1, CTLA4, B7-H3, B7-H4, VISTA, TMIGD2, B7-H7, BTLA, HVEM, CD160, LAG3, TIGIT, CD96, CD155, TIM-3, Galectin-9, Adenosine, Adenosine A2a receptor, IDO, TDO, CEACAM1, SIRP alpha, CD47, CD200R, CD200, OX40, 4-1BB/CD137, GITR, CD28, ICOS, LIGHT, CD27, DNAM-1, 2B4, DC-SIGN, DR3, and CD40. Of these, PD-1, PD-L1, CTLA4, B7-H3, B7-H4, VISTA, TMIGD2, B7-H7, BTLA, HVEM, CD160, LAG3, TIGIT, CD96, CD155, TIM-3, Galectin-9, Adenosine A2a receptor, IDO, TDO, CEACAM1, SIRP alpha, CD47, CD200R and CD200 are inhibitory with regard to immune function; as such, inhibitors of these checkpoint molecules can promote or enhance an anti-tumor immune response. On the other side, OX40, 4-1BB/CD137, GITR, CD28, ICOS, LIGHT, CD27, DNAM-1, 2B4, DC-SIGN, DR3, and CD40 are stimulatory with regard to immune function; as such, agonists of these checkpoint molecules can promote or enhance an anti-tumor immune response.

In one embodiment, an immune modulator polypeptide is a checkpoint modulator polypeptide. In one embodiment, the checkpoint modulator polypeptide is an antagonist, i.e., a polypeptide that inhibits the function of the checkpoint molecule. As noted above, an antagonist of an inhibitory checkpoint molecule will tend to promote an immune response, such as an anti-tumor immune response. The class of immune modulators that are antagonists of inhibitory checkpoint molecules are referred to herein as “checkpoint inhibitors.”

In another embodiment, the checkpoint modulator polypeptide is an agonist, i.e., a polypeptide that upon binding to the checkpoint molecule activates signaling by the checkpoint molecule. As discussed above, when the checkpoint molecule is a stimulatory checkpoint molecule, an agonist will promote an immune response, e.g., an anti-tumor immune response.

In one embodiment, the checkpoint inhibitor polypeptide is or comprises an antibody. antibody reagent, or an antigen-binding fragment thereof that specifically binds to at least one immune checkpoint polypeptide. Non-limiting examples of checkpoint modulators (with checkpoint targets and manufacturers noted in parentheses) can include: MGA271 (B7-H3: MacroGenics); ipilimumab (CTLA-4; Bristol Meyers Squibb); pembrolizumab (PD-1; Merck); nivolumab (PD-1; Bristol Meyers Squibb); atezolizumab (PD-L1; Genentech); galiximab (B7.1; Biogen); IMP321 (LAG3: Immuntep); BMS-986016 (LAG3; Bristol Meyers Squibb); SMB-663513 (CD137 agonist antibody; Bristol-Meyers Squibb); PF-05082566 (CD137 agonist antibody; Pfizer); IPH2101 (KIR; Innate Pharma); KW-0761 (CCR4; Kyowa Kirin); CDX-1127 (CD27 agonist antibody; CellDex); BMS-986178 (Ox40 agonist antibody; Bristol Meyers Squibb); CP-870.893 (CD40 agonist antibody; Genentech); MEDI1873 (GITR agonist antibody; Medimmune); BPS Bioscience antiCD28 agonist antibody; BMS-986226 (ICOS agonist antibody; Bristol Meyers Squibb); tremelimumab (CTLA-4; Medimmune); pidilizumab (PD-1; Medivation); MPDL3280A (PD-L1; Roche); MEDI4736 (PD-L1; AstraZencca); MSB0010718C (PD-L1; EMD Scrono); AUNP12 (PD-1; Aurigene); avelumab (PD-L1; Merck); durvalumab (PD-L1; Medimmune); TSR-022 (TIM3; Tesaro). In one embodiment, a checkpoint inhibitor polypeptide comprises an antibody or antigen-binding reagent thereof that binds PD-L1.

As used herein. “PD-1” or “programmed cell death 1” or “cluster of differentiation 279” or CD279″ is a surface receptor protein that suppresses the immune system expressed by T cells and pro-B cells. PD-1 is encoded by the PDCD-1 gene (Gene ID: 5133). Sequences for PD-1 are known for a number of species, e.g., human PD-L1 mRNA sequences (e.g., NM_005018.2) and polypeptide sequences (e.g., NP_005009.2, SEQ ID NO: 2), as well as murine PD-1 polypeptide sequences (e.g., NP_032824.1, SEQ ID NO: 15), together with any naturally occurring allelic, splice variants, and processed forms thereof.

As used herein. “PD-L1” or “programmed cell death 1 ligand 1” or “cluster of differentiation 274” or CD274″ or “B7 homolog 1” or “B7-H1” is a protein that suppresses the immune system expressed by T cells, natural killer cells, macrophages, myeloid dendritic cells, epithelial cells, B-cells, and vascular endothelial cells. PD-L1 is encoded by the PD-L1 gene (Gene ID: 29126). Sequences for PD-L1 are known for a number of species, e.g., human PD-L1 isoforms a, b, and c mRNA sequences (e.g., the PD-L1 NCBI Reference Sequences are NM_014143.3, NM_001267706.1, NR_052005.1) and polypeptide sequences (e.g., NP_054862.1, SEQ ID NO: 1, 001254635.1, SEQ ID NO: 12, and NP_001300958.1, SEQ ID NO: 13), as well as murine PD-L1 polypeptide sequences (e.g., NP_068693.1, SEQ ID NO: 14), together with any naturally occurring allelic, splice variants, and processed forms thereof.

Binding of PD-L1 to its receptor, PD-1, transmits an inhibitory signal that reduces the proliferation of T cells and can induce apoptosis. Aberrant PD-L1 and/or PD-1 signaling has been shown to promote cancer cell evasion in various tumors. PD-L1/PD-1 blockade can be accomplished by a variety of mechanisms including antibodies that bind PD-1 or its ligand. PD-L1. Examples of PD-1 and PD-L1 blockers are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699; which are incorporated by reference herein in their entiretics. In certain embodiments, the PD-1 inhibitors include anti-PD-L1 antibodies. PD-1 inhibitors include anti-PD-1 antibodies and similar binding proteins such as anti-PD-1 antibody clone RMP1-14, nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224, a fusion protein of B7-DC; an antibody Fc portion; BMS-936559 (MDX-1105-01) for PD-L1 (B7-H1) blockade.

As used herein. “TIGIT” or “T-Cell Immunoreceptor With Ig And ITIM Domains” refers to an immunoglobin protein of the PVR (poliovirus receptor) family encoded by the TIGIT gene. Sequences for TIGIT are known for a number of species, e.g., human TIGIT (the TIGIT NCBI Gene ID is 201633) mRNA sequences (e.g., NM_173799.3), and polypeptide sequences (e.g., NP_776160.2, SEQ ID NO: 6), as well as murine TIGIT polypeptide sequences (e.g., NP_001139797.1, SEQ ID NO: 7), together with any naturally occurring allelic, splice variants, and processed forms thereof. Anti-TIGIT antibodies are known in the art and described herein, for example, in Table 1 and references therein.

Nucleic acids encoding the binding domains of any of the checkpoint modulator antibodies described herein or known in the art can be used to engineer the expression of a checkpoint modulator by a stem cell (e.g., a MSC) as described herein.

As used herein. “CTLA-4” or “cytotoxic T-lymphocyte-associated protein 4” or “CD152” refers to a protein receptor that down regulates immune responses and is constitutively expressed by regulatory T cells. CTLA-4 is encoded by the CTLA-4 gene (Gene ID: 1493). Sequences for CTLA-4are known for a number of species, e.g., human CTLA-4 mRNA sequences (e.g., NM_005214.5), and polypeptide sequences (e.g., NP_005205.2, SEQ ID NO: 3) as well as murine CTLA-4 polypeptide sequences (e.g., NP_033973.2, SEQ ID NO: 8), together with any naturally occurring allelic, splice variants, and processed forms thereof. Anti-CTLA-4 antibodies are known in the art and described herein, for example, in Table 1 and references therein.

As used herein. “TIM-3” or “T-cell immunoglobulin and mucin-domain containing-3” or “Hepatitis A virus cellular receptor” or “HAVCR2” refers to a cell surface protein expressed by CD4+ Th1 and CD8+ Tcl cells that mediates T cell exhaustion and loss of function. TIM-3 is encoded by the HAVCR2 gene (Gene ID: 84868). Sequences for TIM-3 are known for a number of species, e.g., human TIM-3 mRNA sequences (e.g., NM_032782.4), and polypeptide sequences (e.g., NP_116171.3, SEQ ID NO: 4 and SEQ ID NO: 9) as well as murine TIM-3 polypeptide sequences (e.g., NP_599011.2, SEQ ID NO: 10), together with any naturally occurring allelic, splice variants, and processed forms thereof. Anti-TIM-3 antibodies are known in the art and described herein, for example, in Table 1 and references therein.

As used herein, “LAG-3” or “lympocyte-activation gene 3” or “cluster of differentiation 223” or “CD223” refers to a cell surface molecule that negatively regulates cell proliferation, activation, and homeostasis of T cells in a similar mechanism to PD-1 and CTLA-4, LAG-3 is expressed by activated T cells, natural killer cells. B cells, and plasmacytoid dendritic cells. LAG-3 is encoded by the LAG3 gene (Gene ID: 3902). Sequences for LAG-3 are known for a number of species, e.g., human LAG-3 mRNA sequences (e.g., NM_002286.5), and polypeptide sequences (e.g., NP_002277.4, SEQ ID NO: 5) as well as murine LAG-3 polypeptide sequences (e.g., NP_002277.4, SEQ ID NO: 11), together with any naturally occurring allelic, splice variants, and processed forms thereof. Anti-LAG-3 antibodies are known in the art and described herein, for example, in Table 1 and references therein.

In one embodiment, a second genetically modified MSC is engineered to deliver a heterologous polypeptide comprising a cytokine, (e.g., Interleukin (IL)-12B (NCBI Gene ID: 3593), IL-2 (NCBI Gene ID: 3558), IL-5 (NCBI Gene ID: 3567), IL-15 (NCBI Gene ID: 3600), TNF-related apoptosis-inducing ligand (TRAIL; also known as TNF superfamily member 10, TL2, CD253, or TNLG6A; NCBI Gene ID: 8743), an EGFR nanobody-TRAIL fusion, Thrombospondin (THBS)-1 (NCBI Gene ID: 7057), an interferon (e.g., interferon α-1 (NCBI Gene ID: 3439), interferon β-1 (NCBI Gene ID: 3456), or interferon γ (NCBI Gene ID: 3458)), Herpes simplex virus-1 Thymidine kinase (HSV-TK; NCBI Gene ID: 7083), or cytosine deaminase (e.g., E. coli (CD; NCBI Gene ID: 944996). Unless otherwise noted, these polypeptides can refer to human polypeptides, including naturally occurring variants and alleles thereof. In some embodiments of, e.g., in veterinary applications, the polypeptides can refer to the polypeptides of, e.g., dog, cat, cow, horse, pig, and the like. Homologs and/or orthologs of these polypeptides are readily identified for such species by one of skill in the art, e.g., using the NCBI ortholog search function or searching available sequence data for a given species for sequence similar to a given reference sequence.

It is also contemplated that a stem cell (e.g., MSC) can be used to deliver an oncolytic virus, e.g., an oncolytic HSV, adenovirus, or other oncolytic construct. Delivery of oncolytic viruses is further described in Application Nos. PCT/US2013/031,949 and PCT/2014/069,734, which are incorporated herein by reference in their entireties. Non-limiting examples of oncolytic viruses include oncolytic Herpes Simplex Viruses (oHSV), HSV-TRAIL, and oHSV-granulocyte-macrophage colony-stimulating factor (GMCSF).

### T Cells

Aspects of the technology include administering to a subject a population of isolated T cells.

Naïve T cells have not yet encountered their specific antigen. In peripheral lymphoid organs, naïve T lymphocytes can interact with antigen-presenting cells (APCs), which use MHC molecules to present antigen. Once the T cells recognize specific antigens, they proliferate and differentiate into one of several effector T cell subsets. Effector T cells interact with host cells (rather than the pathogen) to carry out their immune function. T cells use co-receptors, e.g., CD4 or CD8 to bind to the MHC molecules.

CD8+ T cells, also known as cytotoxic T cells, mediate direct killing of antigen-presenting target cells. Naive CD8+ T cells are activated upon recognition of antigens presented by MHC class I on dendritic cells in the spleen or lymph nodes. Activated CD8+ T cells expand and become effector CD8+ T cells. CD8+ T cells tend to be evaluated during the study for tumor-infiltrating T cells.

Naive CD4+ T cells recognize antigens presented by major histocompatibility complex (MHC) class II on antigen-presenting cells. Depending on the specific stimuli, the CD4+ T cells can differentiate into various subtypes, including the helper TH1, TH2 and TH17 cells and regulatory T cells (Tregs). A subset of TH2 cells differentiate into allergic disease-related TH2A cells, with a CD45RBlow CD27-phenotype and coexpression of the chemoattractant receptor CRTH2, the natural killer cell marker CD161, and the homing receptor CD49. Exemplary cell surface markers for T helper cells include, INFγ, STAT4, T-bet, Il-4, STAT6, GATA3, TGFβ, FOXP3, Il-17, STAT3, and RORγt.

There are several peripheral subsets CD8⁺ T cells based on the expression of CD45RA and CCR7: a CD45RA⁺ CCR7⁺ subset of naive cells, a CD45RA⁻ CCR7⁺ subset of antigen-experienced memory T cells, a CD45RA⁻ CCR7⁻ effector memory cell subset, and a CD45RA⁺ CCR7⁻ subset of differentiated, antigen-experienced effector cells. Also, there are effector memory CD8⁺ T cells expressing CD69 and CD103 and residing in non-lymphoid tissues. A subpopulation of CD8⁺ T cells shows a memory cell phenotype: CD62L⁻/⁺ CCR7⁻ CD27⁻/⁺. Activated cytotoxic CD8⁺ T cells downregulate expression of L-selectin and CCR7 and upregulate surface expression of CD44, LFA-1 and/or α4β1 integrin. In addition, there are CD8⁻ cytotoxic T cells: CD4⁺ cytotoxic T cells and gamma delta T cells.

Memory T cells vary in their surface receptor expression, effector and trafficking abilities. There are four major subsets of memory T cells: central memory, effector memory, tissue-resident memory and stem memory T cells. Multiple signals regulate the differentiation of CD4+ T cells into central and peripheral memory cells. CD4+ T central memory cells express CD62L and CCR7, which are important for their migration. The peripheral T stem cell memory cells express CXCR3 and CD95 molecules. In addition, both naive and memory T-cell subsets express a variety of functional molecules, which are described herein below in Table 3.

TABLE 3
Functional molecules expressed on naive and memory T cells

| Process | Antigen | Function |
|---|---|---|
| Costimulation/ | CD27 | Costimulation |
| Survival | CD28 | Costimulation |
| | CD127 | IL-7 signaling |
| | PD-1 | Inhibition of effector function |
| | CD122 | IL-2/IL-15 signaling |
| | CD132 | γc cytokine signaling |
| | KLRG-1 | Inhibition of effector function |
| Activation | HLA | DR Peptide presentation |
| | CD38 | Calcium flux/signal transduction |
| | CD69 | Proliferation |
| Adhesion | CD11a | Adhesion to APC/endothelium |
| | CD58 | Adhesion to APC |
| | CD99 | Transendothelial migration |
| Migration | CD62L | Secondary lympboid tissues homing |
| | CD103 | Gut homing |
| | CCR4 | Chemokine response/T_{H}2 associated |
| | CCR5 | Homing to inflamed tissues |
| | CCR6 | Chemokine response/T_{H}17 associated |
| | CCR9 | Gut homing |
| | CCR10 | Skin homing |
| | CXCR3 | Homing to inflamed tissues |
| | CXCR4 | Homing to bone marrow |
| | CLA | Skin homing |
| Cytolytic | Granzyme A | Cleavage of cellular proteins |
| molecules | Granzyme B | Cleavage of cellular proteins |
| | Perforin | Pore-forming |
| Miscellaneous | CD161 | Regulation of proliferation/cytotoxicity |
| | IL-18Ra | 18Ra Response to IL-18 |
| | c-Kit | Response to SCF |
| | CD130 | Response to IL-6 |

The majority of T cells bear α and β chains in their T cell receptor (TCR). However, there is a population of T cells which have TCR formed by γ and δ chains. These cells, gamma delta T cells, bind to BTN2A1 and BTN3A1, and are significantly enriched in epithelia. Gamma delta T cells regulate immune responses by various mechanisms, including suppression of effector T cell and TH1 cell functions, blockage of neutrophil influx and regulation of antigen-presenting cell activity.

Tumor infiltrating T cells are a subpopulation of T cells that are capable of infiltrating or are found within a solid tumor. Exemplary subsets of tumor infiltrating T cells are described herein below in Table 4.

TABLE 4
Exemplary subsets of tumor infiltrating T cells

| T cellular | | |
|---|---|---|
| subsets | Markers | Functions |
| Cytotoxic | CD8⁺ | Exhibit cytotoxicity against tumor |
| T cells | | cells. |
| CD4⁺ T_{H}9 | CD4⁺ | Secrete IL-9, IL-10 and IFNg and |
| T helpers | | inhibit tumor growth. |
| CD4⁺ Tregs | CTLA-4⁺ GITR⁺ PD-1⁺ CCR⁺ | Suppress anti-tumor immune |
| | CCR4⁺ CXCR4⁺ GITR⁺ | response. |
| | LAG3⁺ OX40⁺ ICOS⁺ | |
| CD8⁺ Mucosal- | Rearranged TCRβ chains with | Production of T_{H}1-mediated |
| associated | Vβ gene segments | cytokines and direct lysis of |
| invariant T cells | | neoplastic cells. |
| CD8⁺ memory | CD95⁺ CD45RO^{hi} and CD95⁺ | Are associated with better |
| | CD45RO^{low} subsets | prognosis and longer survival in |
| | | patients with breast tumors. |
| γδ T cells | Vδ1 and Vγ9Vδ2 | Have anti-tumor properties by |
| | | differentiating into cytotoxic T |
| | | cells. |

Anti-tumor effects can be mediated, for example, via the differentiation of tumor-infiltrating T cells or other T cells into cytotoxic T cells. Vδ1 and Vγ9Vβ2 T cells are two populations of γδ T cells, which can be present in the tumor microenvironment. Both subsets develop cytotoxic capabilities. The lysis of target tumor cells by γδ T cells is mediated via different mechanisms involving, for example, granzyme B, perforin and Fas ligand. In addition, γδ T cells can induce cytostatic effects by producing IFN-γ or TNF-α.

TH9 cells are known to secrete IL-9 and IL-10 and inhibit tumor growth, and some TH9 cells can secrete IFNγ. TH9 cells, which have been shown to infiltrate, for example, colorectal and lung tumors, may be regulated through the PD-1/PD-L1 pathway and may stimulate the proliferation of CD8+ cells. Expansion of TH9 cells is usually accompanied by an increase of IL-9+ IL-4− and IL-9+ IL-4+ cell subsets.

The standard methods for measurement of T cell immune responses include Enzyme-Linked Immuno Spot assay (ELISpot), Intracellular Cytokine Staining assay (ICS), Tetramer assay and Flow Cytometry. The ELISpot and ICS assays apply in vitro stimulation to analyze the cytokine expression profiles of responding cells. The ELISpot method detects spots of cytokines secreted by individual cells, and ICS examines surface markers and produced cytokines. Multiple approaches can measure the proliferation of T cells in response to specific antigens, including thymidine incorporation assay, flow cytometric analysis of CD38 expression or ELISA detection of BrdU incorporation into DNA of proliferating T cells.

In one embodiment, the polypeptide expressed on the surface of a T cell that is bound by a binding domain of a MiTE is CD3 or CD38, CD28, 41BB or OX40.

While a goal of the methods described herein is to bring T cells into the tumor microenvironment that are or can be activated to a cytotoxic effector phenotype, it is contemplated that the polypeptide expressed on the surface of a T cell that is bound by a binding domain of a MiTE could alternatively be the T cell receptor or a component thereof. This approach could be appropriate where, for example, binding of the MiTE's T cell binding domain itself activates the receptor, or, for example, where binding of the MiTE's T cell binding domain does not interfere with signaling by the T cell receptor. The T cell receptor (TCR) is a heterodimeric receptor molecule found on the surface of a T cell that recognizes and binds an antigen bound to/displayed upon the MHC or an APC. Binding of MHC-displayed antigen by the TCR initiates signal transduction by the TCR necessary for activation of the T cell.

For a T cell to be fully activated, the T cell is generally co-stimulated (e.g., receiving simultaneous first and second signaling via binding of an antigen-specific and antigen non-specific molecule, respectively). In a similar manner to the embodiment discussed above, then, it is contemplated that the polypeptide expressed on the surface of a T cell that is bound by a binding domain of a MiTE could alternatively be a T cell co-stimulatory molecule. A co-stimulatory molecule is the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule. BTLA, a Toll-like receptor, CD27, CD28, 4-1BB, OX40, CD30, CD40, PD-1, ICOS, lymphocyte function-associated antigen-1 (LFA-1), CD2, CD7, LIGHT, NKG2C, B7-H3, and CD83. This approach could be appropriate where, for example, the binding of the MiTE's T cell binding domain either activates the co-stimulatory signaling, or, alternatively, does not interfere with signaling via the co-stimulatory molecule.

### T Cell Expansion and Culturing

In another embodiment, the method comprises isolating T cells and expanding them in culture. In another embodiment, the invention further comprises cryopreserving the T cells prior to or after expansion.

T cells can be isolated from peripheral blood mononuclear cells (PBMC) by either positive or negative selection based on antibody capture. Positive selection strategies use antibodies that bind markers expressed on T cells to directly bind and isolate the T cells. Negative selection strategies use a cocktail of antibodies that bind and remove PBMC cells other than T cells, to leave the T cells as the enriched population. While either approach can be used, negative selection can leave the T cells in a more natural state, as their surface markers have not been bound, thereby limiting potential signaling provoked by such binding. Invitrogen/ThermoFisher Scientific sells a negative selection human Tcell isolation kit, the DYNABEADS™ UNTOUCHED™ Human T cells Kit (Cat. No. 11344D). If, on the other hand, binding to the T cell markers assists in activating the T cells to a proliferative state, positive selection can be advantageous. Selection based on antibody binding to, e.g., CD3 or CD28 can promote activation at the same time as positively selecting the T cell population from the larger PBMC population.

In one embodiment, the T cell population is expanded prior to administration. Methods for T cell expansion are known in the art, e.g., as described in US Patent Application 2019/0270966A1; 2018/0312848A1; and U.S. Pat. No. 10,286,066B2. For example, T cells can be incubated in cell culture medium in a culture apparatus for a period of time or until the cells reach confluence or high cell density for optimal passage before passing the cells to another culture apparatus. The culturing apparatus can be of any culture apparatus commonly used for culturing cells in vitro. For maintenance or expansion passaging. T cells can be plated at, e.g., 4.5×10⁵ cells/ml. The cells generally double every 28-30 hours, with passaging back to 4.5×10⁵ cells/ml performed, for example, every other day. Higher or lower plating and/or passage densities can be used. The T cells are then harvested from the culture apparatus whereupon the T cells can be used immediately or cryopreserved to be stored for use at a later time. In one embodiment, the methods as described include cryopreserving the expanded T cells.

Optimized methods for γδ T cell expansion are known in the art, e.g., as described in Wang, R-N., et al. Mol Med Rep. 2019 March; 19(3):1471-1480, doi: 10.3892/mmr.2019.9831. Epub 2019 Jan. 8, the contents of which are incorporated herein by reference.

Another procedure for ex vivo expansion of T cells is described in U.S. Pat. No. 5,199,942 (incorporated herein by reference). Briefly, ex vivo culture and expansion of T cells comprises the addition, to the cellular growth medium, of factors such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

Conditions appropriate for T cell culture include an appropriate medium (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α, or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the T cells can include an agent that can co-stimulate the T cells. For example, an agent that can stimulate CD3 is an antibody to CD3, and an agent that can stimulate CD28 is an antibody to CD28. Other co-stimulatory molecules include, for example, CD27, CD83, CD86 and CD127, among others. In one embodiment, the T cells expand in the range of about 20 fold to about 50 fold, or more by culturing the population.

### T Cell Activation

In some embodiments, the administered T cells are activated T cells. As used herein, the term “activation” can refer to the state of a T cell that has been sufficiently stimulated to induce detectable cellular proliferation. In some embodiments activation can refer to induced cytokine production. In other embodiments, activation can refer to detectable effector functions. At a minimum, an “activated T cell” as used herein is a proliferative T cell. In one embodiment, an activated T cell can be assessed by its cell-surface molecule profile. Non-limiting examples of molecules expressed the surface of an activated T cell include CD25, 4-1BB, and HLA-DR. Activated T cells also secrete cytokines, including, but not limited to IL-2. Methods to identify these surface molecules and secreted cytokines are known in the art.

In one embodiment, the T cell is activated prior to administration. T cell activation occurs through simultaneous engagement of the T cell receptor and co-stimulatory molecules (i.e., CD3 and CD28). This results in the activation of downstream signaling pathways (e.g., PI3K signaling), and eventual immune response (involving cytokine production). Following activation, a T cell expresses a variety of proteins (also known as markers), including, but not limited to CD69, CD71, CD25, and HLA-DR. In addition, an activated T cell has an altered cell surface protein glycosylation profile.

In one embodiment, following activation the T cell population increases at least 1-fold, at least 2-fold, at least 3-fold, at least 4-fold, at least 5-fold, at least 6-fold, at least 7-fold, at least 9-fold, or at least 10-fold or more. One skilled in the art will be capable of measuring T cell growth, for example, via a T cell expansion curve. Briefly, T cell volumes are measured at days 3, 5, 7, and 9 post activation protocol using, e.g., a Multisizer™ 3 Coulter Counter (Beckman Coulter). Cellular viability can be determined by staining, e.g., with Acridine Orange/Propidiumlodide exclusion dye using a Luna-FL™ Cell Counter (Logos Biosystems).

One skilled in the art will be capable of determining if a T cell has become activated. For example, one can use a Proliferative Capacity assay (CFSE dilution and absolute T cell numbers are assessed by FACS using fluorescently-labeled counting beads), a Cytokine Production assay (10-Plex Luminex Assays using cytokine levels as a readout), a Target-cell Killing Capacity assay (Bioluminescence analysis of target cells in vitro or animal model system to track both tumor and engineered T cells infused in immunodeficient mice), and/or a Cell Degranulation Analysis (CD107a release assay in response to target cells as measured by FACS). One skilled in the art can additionally determine if a T cell is activated by assessing the markers present on the T cell surface, or by examining the glycosylation profile of the cell surface.

### Local Cell Delivery and Matrix Encapsulation

Local delivery of cells, whether genetically modified stem cells (e.g., MSCs) as described herein or T cells as described herein, can provide benefits for cancer therapy. In one aspect, local delivery can provide a high local concentration of the therapeutic polypeptide(s) or effector cells. However, one of the benefits of stem cell (e.g., MSCs) to deliver therapeutic polypeptides to the tumor microenvironment is their natural tumor-homing activity. These benefits of systemic administration can be hampered for certain tumor types, notably brain tumors, where the blood-brain barrier can limit access of systemically administered cells to a tumor. For this reason, local delivery to the site of a tumor, and especially considering the immunostimulatory effects of tumor resection demonstrated herein, local delivery of therapeutic cells to the site of tumor resection, can be of particular benefit for the treatment of brain tumors, including but not limited to GBM, which are notoriously difficult to treat.

In one embodiment, the genetically modified stem cells (e.g., MSCs) are encapsulated in a matrix. In another embodiment, the T cells are encapsulated in a matrix. In another embodiment, both the MSCs and the T cells are encapsulated in a matrix. This can assist in retaining stem cells (e.g., MSCs) and/or T cells in a given location, such as a tumor resection cavity. The matrix can minimize wash out of cells from the resection cavity, e.g., by CSF in the case of brain tumors. As such, cells encapsulated in or associated with a matrix as described herein are formulated for delivery to a tumor resection caity.

A matrix useful in the methods and compositions described herein will permit MSCs and/or T cells to migrate away from the matrix, rather than containing the cells within the matrix permanently. As used herein, “matrix” refers to a biological material that comprises a “biocompatible substrate” that can be used as a material that is suitable for implantation into a subject or into which a cell population can be deposited. A biocompatible substrate does not cause toxic or injurious effects once implanted in the subject. The biocompatible substrate can but need not necessarily provide the supportive framework that allows cells to attach to it, and grow on it. Cultured populations of cells (e.g., genetically modified stem cells (e.g., MSCs) and/or T cells) can be prepared with the biocompatible substrate (i.e., the matrix), which provides the appropriate interstitial distances required, e.g., for cell-cell interaction. As used herein, “encapsulated” refers to a cell that is enclosed within the matrix.

A matrix can be used to aid in further controlling and directing a cell or population of genetically modified stem cells (e.g., MSCs) and/or T cells as described herein. A matrix can be designed or selected to provide environmental cues to control and direct the migration of cells to a site of injury or disease. A structure can be engineered from a nanometer to micrometer to millimeter to macroscopic length, and can further comprise or be based on factors such as, but not limited to, material mechanical properties, material solubility, spatial patterning of bioactive compounds, spatial patterning of topological features, soluble bioactive compounds, mechanical perturbation (cyclical or static strain, stress, shear, etc.), electrical stimulation, and thermal perturbation.

In one embodiment, the matrix comprises a synthetic matrix. In one embodiment, the matrix comprises a thiol-modified hyaluronic acid and a thiol-reactive cross-linker molecule. In one embodiment, the thiol-reactive cross-linker molecule is polyethylene glycol diacrylate. Further description of components useful in constructing a matrix, as well as instruction for making a matrix, can be found in U.S. patent application Ser. No. 15/225,202, which is incorporated herein in its entirety by reference.

Methods of encapsulation of stem cells are known in the art and can be found, for example, in Shah et al. Biomatter. 2013 and Kauer et al. Nature Neuroscience. 2013.

For example, the synthetic extracellular matrix (ECM) components, such as those from Hystem and Extralink (Glycosan Hystem-C, Biotime Inc.), can be reconstituted according to the manufacturer's protocols. Stem cells (e.g. 1×10⁵, 2×10⁵ or 4×10⁵ cells) can be re-suspended in Hystem (e.g. 14 μl) and the matrix is cross-linked by adding Extralink (e.g. 6 μl). After about 20 minutes (gelation time) at 25° C., the stem cell and ECM hydrogel can be placed in the center of different sizes (35 or 60 mm) of glass-bottomed dish. Bioluminescence imaging can be used to determine the viability of the genetically modified stem cells (e.g., MSCs) and/or T cells expressing a detectable label. To assess the numbers of cells expressing immunomodulatory polypeptides and the amounts of such polypeptides expressed, methods known in the art can be used such as flow cytometry, Western blotting, immunohistochemistry, or enzyme-linked immunosorbent assay (ELISA).

### Detectable Labels

For tracking purposes, cells (e.g., genetically modified stem cells (e.g., MSCs), and/or T cells) can be tagged with a detectable label. As used herein, the term “detectable label” refers to a composition capable of producing a detectable signal indicative of the presence of a target. Detectable labels include any composition detectable by spectroscopic, photochemical, biochemical, immunochemical, electrical, optical or chemical means. Suitable labels include fluorescent molecules, radioisotopes, nucleotide chromophores, enzymes, substrates, chemiluminescent moieties, bioluminescent moieties, and the like. As such, a label is any composition detectable by spectroscopic, photochemical, biochemical, immunochemical, electrical, optical or chemical means needed for the methods and devices described herein.

A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound.

Other exemplary detectable labels include luminescent and bioluminescent markers (e.g., biotin, luciferase (e.g., bacterial, firefly, click beetle and the like), luciferin, and acquorin), radiolabels (e.g., 3H, 125I, 35S, 14C, or 32P), enzymes (e.g., galactosidases, glucorinidases, phosphatases (e.g., alkaline phosphatase), peroxidases (e.g., horseradish peroxidase), and cholinesterases), and calorimetric labels such as colloidal gold or colored glass or plastic (e.g., polystyrene, polypropylene, and latex) beads. Patents teaching the use of such labels include U.S. Pat. Nos. 3,817,837, 3,850,752, 3,939,350, 3,996,345, 4,277,437, 4,275,149, and 4,366,241, each of which is incorporated herein by reference.

Means of detecting such labels are well known to those of skill in the art. Thus, for example, radiolabels can be detected using photographic film or scintillation counters, fluorescent markers can be detected using a photo-detector to detect emitted light. Enzymatic labels are typically detected by providing the enzyme with an enzyme substrate and detecting the reaction product produced by the action of the enzyme on the enzyme substrate, and calorimetric labels can be detected by visualizing the colored label.

### Cancer

Cancer involves the proliferation of cells that have lost normal cellular control, resulting in unregulated growth, lack of differentiation, local tissue invasion, and metastasis. Cancers are classified based on the histological type (e.g., the tissue in which they originate) and their primary site (e.g., the location of the body the cancer first develops), and can be a carcinoma, a melanoma, a sarcoma, a myeloma, a leukemia, or a lymphoma. “Cancer” can also refer to a solid tumor. As used herein, the term “tumor” refers to an abnormal growth of cells or tissues, e.g., of malignant type or benign type. “Cancer” can be metastatic, meaning the cancer cells have disseminated from a primary site of origin and migrated to a secondary site.

In one embodiment, the cancer treated herein is a sarcoma.

Sarcomas are cancers that originate in supportive and connective tissues, for example bones, tendons, cartilage, muscle, and fat. Sarcoma tumors usually resemble the tissue in which they grow. Non-limiting examples of sarcomas include, Osteosarcoma or osteogenic sarcoma (originating from bone), Chondrosarcoma (originating from cartilage), Leiomyosarcoma (originating from smooth muscle), Rhabdomyosarcoma (originating from skeletal muscle), Mesothelial sarcoma or mesothelioma (originate from membranous lining of body cavities), Fibrosarcoma (originating from fibrous tissue), Angiosarcoma or hemangioendothelioma (originating from blood vessels), Liposarcoma (originating from adipose tissue), Glioma or astrocytoma (originating from neurogenic connective tissue found in the brain), Myxosarcoma (originating from primitive embryonic connective tissue), or Mesenchymous or mixed mesodermal tumor (originating from mixed connective tissue types).

In one embodiment, the cancer is a glioblastoma.

In alternative embodiment, the cancer treated herein is a carcinoma, a melanoma, a sarcoma, a myeloma, a leukemia, a lymphoma, or a solid tumor.

Leukemias (also known as “blood cancers”) are cancers of the bone marrow, which is the site of blood cell production. Leukemia is often associated with the overproduction of immature white blood cells. Immature white blood cells do not function properly, rendering the patient prone to infection. Leukemia additionally affects red blood cells, and can cause poor blood clotting and fatigue due to anemia.

In one embodiment of any aspect, the leukemia is acute myeloid leukemia (AML), Chronic myeloid leukemia (CML), Acute lymphocytic leukemia (ALL), and Chronic lymphocytic leukemia (CLL). Examples of leukemia include, but are not limited to, Myelogenous or granulocytic leukemia (malignancy of the myeloid and granulocytic white blood cell series), Lymphatic, lymphocytic, or lymphoblastic leukemia (malignancy of the lymphoid and lymphocytic blood cell series), and Polycythemia vera or erythremia (malignancy of various blood cell products, but with red cells predominating)

A carcinoma is a cancer that originates in an epithelial tissue. Carcinomas account for approximately 80-90% of all cancers. Carcinomas can affect organs or glands capable of secretion (e.g., breasts, lung, prostate, colon, or bladder). There are two subtypes of carcinomas: adenocarcinoma, which develops in an organ or gland, and squamous cell carcinoma, which originates in the squamous epithelium. Adenocarcinomas generally occur in mucus membranes, and are observed as a thickened plaque-like white mucosa. They often spread easily through the soft tissue where they occur. Exemplary adenocarcinomas include, but are not limited to, lung cancer, prostate cancer, pancreatic cancer, esophageal cancer, and colorectal cancer. Squamous cell carcinomas can originate from any region of the body. Examples of carcinomas include, but are not limited to, prostate cancer, colorectal cancer, microsatellite stable colon cancer, microsatellite instable colon cancer, hepatocellular carcinoma, breast cancer, lung cancer, small cell lung cancer, non-small cell lung cancer, lung adenocarcinoma, melanoma, basal cell carcinoma, squamous cell carcinoma, renal cell carcinoma, ductal carcinoma in situ, ductal carcinoma.

Melanoma is a type of cancer forming from pigment-containing melanocytes. Melanoma typically develops in the skin, but can occur in the mouth, intestine, or eye.

Myelomas are cancers that originate in plasma cells of bone marrow. Non-limiting examples of myelomas include multiple myeloma, plasmacytoma and amyloidosis.

Lymphomas develop in the glands or nodes of the lymphatic system (e.g., the spleen, tonsils, and thymus), which purifies bodily fluids and produces white blood cells, or lymphocytes. Unlike leukemia, lymphomas form solid tumors. Lymphoma can also occur in specific organs, for example the stomach, breast, or brain; this is referred to as extranodal lymphomas). Lymphomas are subclassified into two categories: Hodgkin lymphoma and Non-Hodgkin lymphoma. The presence of Reed-Sternberg cells in Hodgkin lymphoma diagnostically distinguishes Hodgkin lymphoma from Non-Hodgkin lymphoma. Non-limiting examples of lymphoma include Diffuse large B-cell lymphoma (DLBCL), Follicular lymphoma, Chronic lymphocytic leukemia (CLL), Small lymphocytic lymphoma (SLL), Mantle cell lymphoma (MCL), Marginal zone lymphomas, Burkitt lymphoma, hairy cell leukemia (HCL). In one embodiment, the cancer is DLBCL or Follicular lymphoma.

In one embodiment, the cancer is a solid tumor. Non-limiting examples of solid tumors include Adrenocortical Tumor, Alveolar Soft Part Sarcoma, Chondrosarcoma, Colorectal Carcinoma, Desmoid Tumors, Desmoplastic Small Round Cell Tumor, Endocrine Tumors, Endodermal Sinus Tumor, Epithelioid Hemangioendothelioma, Ewing Sarcoma, Germ Cell Tumors (Solid Tumor), Giant Cell Tumor of Bone and Soft Tissue, Hepatoblastoma, Hepatocellular Carcinoma, Melanoma, Nephroma, Neuroblastoma, Non-Rhabdomyosarcoma Soft Tissue Sarcoma (NRSTS), Osteosarcoma, Paraspinal Sarcoma, Renal Cell Carcinoma, Retinoblastoma, Rhabdomyosarcoma, Synovial Sarcoma, and Wilms Tumor. Solid tumors can be found in bones, muscles, or organs, and can be sarcomas or carcinomas.

In one embodiment, the solid tumor is a primary tumor.

In one embodiment, the solid tumor is a metastatic tumor.

In one embodiment of any aspect, the cancer is not resistant to a cancer therapy.

In one embodiment of any aspect, the cancer is resistant to a cancer therapy. A cancer resistant to a therapy, is one that previously responded to the treatment but is now capable of growing or persisting despite the presence of continued treatment. Resistance to a therapy can occur due to, e.g., acquired mutations in the cancer cell, gene amplification in the cancer cell, or the cancer cell develops mechanisms to prevent the uptake of the treatment. In one embodiment of any aspect, the cancer is not resistant to a cancer therapy.

In one embodiment, the cancer is metastatic (e.g., the cancer has disseminated from its primary location to at least one secondary location).

In one embodiment, the cancer, e.g., a solid tumor, has been resected prior to administration. In one embodiment, where the tumor is resected, the cells as described herein can be placed in the tumor resection cavity.

In one embodiment, the cancer, e.g., a solid tumor, has been resected following administration.

In one embodiment, the cancer, e.g., a solid tumor, will be resected prior to administration.

In one embodiment, the cancer, e.g., a solid tumor, will be resected following administration.

In one embodiment, the cancer has relapsed following administration of a cancer therapy. A “relapsed cancer” is defined as the return of a disease or the signs and symptoms of a disease after a period of improvement.

### Administration

In some embodiments, the methods described herein relate to treating a subject having or diagnosed with a solid tumor cancer by administering a genetically modified stem cell a described herein, and an isolated T cell population. In one embodiment, the solid tumor has been resected prior to administration. Subjects having a condition (e.g., glioblastoma or other solid tumor cancer) can be identified by a physician using current methods of diagnosing the condition. Symptoms and/or complications of the condition, which characterize these conditions and aid in diagnosis are well known in the art and include but are not limited to, fatigue, persistent infections, and persistent bleeding. Tests that may aid in a diagnosis of the condition include, but are not limited to blood screening and imaging (e.g., PET scan), and are known in the art for a given condition. A family history for a condition, or exposure to risk factors for a condition can also aid in determining if a subject is likely to have the condition or in making a diagnosis of the condition.

In one embodiment, the genetically modified stem cells (e.g., MSCs) and (non-genetically modified) T cells are administered at substantially the same time. Substantially contemporaneous administration can facilitate recruitment of the administered T cells to the tumor cells by the MiTE expressed by the modified MSCs. In one embodiment, the genetically modified stem cells (e.g., MSCs) are administered prior to administration of the T cells. In one embodiment, the genetically modified stem cells (e.g., MSCs) are administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 mins; or 1 2, 3, 4, 5, 6, 7, 8, 9, 10, 11, 12, 13, 14, 15, 16, 17, 18, 19, 20, 21, 22, 23 hours; or 1, 2, 3, 4, 5, days prior to administration of the T cells.

Similarly, in one embodiment, the genetically modified stem cells (e.g., MSCs) are administered following administration of the T cells. In one embodiment, the genetically modified stem cells (e.g., MSCs) are administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 mins; or 1 2, 3, 4, 5, 6, 7, 8, 9, 10, 11, 12, 13, 14, 15, 16, 17, 18, 19, 20, 21, 22, 23 hours; or 1 or 2 days following administration of the T cells.

In one embodiment, the genetically modified stem cells (e.g., MSCs) described herein are administered directly into the cavity formed by resection of a tumor, and the T cells described herein are administered systemically. In another embodiment, the MSCs and the T cells are administered directly into the cavity formed by resection of the tumor at substantially the same time. In another embodiment, the MSCs and the T cells are administered systemically at substantially the same time, or at different time points. In yet another embodiment, the MSC are administered systemically, and the T cells are administered directly into the cavity formed by resection of the tumor. The natural tumor-homing activity of MSCs can assist in the tumor-localization of systemically-administered MSCs.

In one embodiment, the stem cells are not administered systemically.

While MSCs and NSCs can cross the blood-brain barrier, in one embodiment, the stem cells are administered intracerebroventricularally, such that they bypass the blood brain barrier.

The compositions described herein can be administered to a subject having or diagnosed as having a condition. In some embodiments, the methods described herein comprise administering an effective amount of activated genetically modified stem cells, and T cells as described herein to a subject in order to alleviate a symptom of the condition. As used herein. “alleviating a symptom of the condition” is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. In one embodiment, the compositions described herein are administered systemically or locally. In a preferred embodiment, the compositions described herein are administered intravenously. In another embodiment, the compositions described herein are administered at the site of the tumor or tumor resection.

The term “effective amount” as used herein refers to the amount of genetically modified stem cells (e.g., MSCs) and T cells needed to alleviate at least one or more symptom of the disease (e.g., glioblastoma or other caner, including solid tumor cancer), and relates to a sufficient amount of the cell preparation or composition to provide the desired effect. The term “therapeutically effective amount” therefore refers to an amount of genetically modified stem cells (e.g., MSCs) and T cells that is sufficient to provide a particular anti-cancer effect when administered to a typical subject. An effective amount as used herein, in various contexts, would also include an amount sufficient to delay the development of a symptom of the disease, alter the course of a symptom disease (for example but not limited to, slowing the progression of a condition), or reverse a symptom of the condition. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be evaluated by standard pharmaceutical procedures in cell cultures or experimental animals. The dosage can vary depending upon the dosage form employed and the route of administration utilized.

While stem cells (e.g., MSCs) will most often be administered locally and T cells systemically, in one aspect, the technology described herein relates to a pharmaceutical composition comprising activated T cells and modified MSCs as described herein, and optionally a pharmaceutically acceptable carrier. The active ingredients of such a pharmaceutical composition at a minimum comprise T cells and genetically modified MSCs as described herein. In some embodiments, the active ingredients of the pharmaceutical composition consist essentially of T cells and genetically modified MSCs as described herein. In some embodiments, the active ingredients of the pharmaceutical composition consist of T cells and genetically modified MSCs as described herein. Pharmaceutically acceptable carriers for cell-based therapeutic formulations are known in the art and can include, for example, saline and aqueous buffer solutions. Ringer's solution, and serum components, such as serum albumin, among others. Carriers for parenteral dosage forms of T cells and genetically modified stem cells (e.g., MSCs cells) as described herein can also include, without limitation: glucose/dextrose solution; aqueous vehicles including but not limited to, sodium chloride injection, Ringer's injection, lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Carriers or excipients for such a composition can also include a matrix as described herein. The terms such as “excipient”, “carrier”, “pharmaceutically acceptable carrier” or the like are used interchangeably herein.

In some embodiments, the pharmaceutical composition comprising T cells and genetically modified stem cells (e.g., MSCs, NSCs) as described herein can be a parenteral dose form. Since administration of parenteral dosage forms typically bypasses the patient's natural defenses against contaminants, the components apart from the T cells and genetically modified MSCs themselves are preferably sterile or capable of being sterilized prior to administration to a patient. Examples of parenteral dosage forms include, but are not limited to, solutions or suspensions in a pharmaceutically acceptable vehicle ready for injection, and emulsions. Any of these can be added to the preparation of T cells and genetically modified MSCs prior to administration.

### Dosage

A pharmaceutical composition comprising the T cells and genetically modified stem cells (e.g., MSCs cells) described herein can generally be administered at a dosage of 10⁶ to 10¹² cells per administration. Cells administered systemically can be administered in higher numbers than cells administered locally. In some instances, cells can be administered at 10⁵ to 10⁸ cells/kg body weight, including all integer values within that range. If necessary, T cells and/or genetically modified stem cells (e.g., MSCs cells) can also be administered multiple times at these dosages. Where indicated, the cells can be administered by using infusion techniques that are commonly known in immunotherapy (see, e.g., Rosenberg et al., New Eng. J. of Med. 319:1676, 1988).

“Unit dosage form” as the term is used herein refers to a dosage suitable for one administration. By way of example a unit dosage form can be an amount of therapeutic disposed in a delivery device, e.g., a syringe or intravenous drip bag. In one embodiment, a unit dosage form is administered in a single administration. In another embodiment, more than one unit dosage form can be administered simultaneously.

Modes of administration can include, for example intravenous (i.v.) injection or infusion. The compositions described herein can be administered to a patient transarterially, intratumorally, intranodally, or intramedullary. In some embodiments, the compositions of genetically modified stem cells (e.g., MSCs cells) and T cells described herein can be injected directly into a tumor, lymph node, or site of infection. In one embodiment, the compositions described herein are administered into a body cavity or body fluid (e.g., ascites, pleural fluid, peritoneal fluid, or cerebrospinal fluid).

The dosage of the above treatments to be administered to a patient will vary with the precise nature of the condition being treated and the recipient of the treatment. The scaling of dosages for human administration can be performed according to art-accepted practices.

In some embodiments, a single treatment regimen is required. In others, administration of one or more subsequent doses or treatment regimens can be performed. In some embodiments, no additional treatments are administered following the initial treatment. In one embodiment, T cells are administered once, and genetically modified stem cells (e.g., MSCs cells) are administered at least one additional time. In one embodiment, genetically modified MSCs are administered once, and T cells are administered at least one additional time.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

### Kits

Included in the present technology are kits comprising at least one composition of matter as described herein and a matrix as described herein, together with packaging materials therefor, optionally with instructions for use. The kit can comprise a genetically modified MSC or NSC as described herein, including but not limited to an off-the-shelf or allogeneic (to the recipient) genetically modified MSC or NSC, and a matrix as described herein. The kits can also include a prodrug as described herein. For example, in one embodiment, the present technology provides a kit comprising: (i) a composition comprising a first stem cell engineered to express a multispecific T cell engager polypeptide, (ii) a pharmaceutical carrier as described herein, (iii) a matrix, and optionally (iv) a prodrug and packaging and optionally instructions for use. A kit of the technology can optionally comprise at least one additional reagent (e.g., standards, markers and the like). Kits typically include a label indicating the intended use of the contents of the kit. Associated with such a kit can be a notice in the form prescribed by a governmental agency regulating the manufacture, use or sale of pharmaceuticals or biological products, which notice reflects approval by the agency of manufacture, use or sale for human administration.

Engineered stem cells of a kit as described herein can be off-the-shelf mesenchymal stem cells, or stem cells obtained from a subject.

The kit can further comprise reagents and other tools for detecting a cell type (e.g. a tumor cell) in a sample or in a subject, or for diagnosing whether a patient belongs to a group that responds to a therapeutic strategy which makes use of a compound, composition, or related method of the present technology, e.g., such as a method described herein.

The technology described herein can further be described in the following numbered paragraphs.
1. A method of treating cancer, the method comprising administering to a subject in need thereof:
   a) a stem cell engineered to secrete a multispecific T cell engager (MiTE) polypeptide construct; and
   b) an isolated population of T cells that are autologous to the subject.
2. The method of paragraph 1, wherein the stem cell has a tumor tropism.
3. The method of any of the preceding paragraphs, wherein the stem cell is a mesenchymal stem cell (MSC) or a neuronal stem cell (NSC).
4. The method of any of the preceding paragraphs, wherein the stem cell is derived from an induced pluripotent stem cell.
5. The method of any of the preceding paragraphs, wherein the isolated population of T cells is enriched for γδ T cells.
6. The method of any of the preceding paragraphs, wherein the isolated population of T cells is not genetically manipulated.
7. The method of any of the preceding paragraphs, wherein the isolated T cell population is expanded in culture prior to administering.
8. The method of any of the preceding paragraphs, wherein the MiTE polypeptide comprises a binding domain that specifically binds a polypeptide expressed on the surface of a cancer cell and a binding domain that specifically binds a polypeptide expressed on the surface of a T cell.
9. The method of any of the preceding paragraphs, wherein the polypeptide expressed on the surface of a cancer cell is selected from the group consisting of EGFR, EGFRvIII, HER2, IL-13Rα2, EphA2 and GD2.
10. The method of any of the preceding paragraphs, wherein the polypeptide expressed on the surface of a T cell is selected from the group consisting of CD3, CD28, 41BB and OX40.
11. The method of any of the preceding paragraphs, wherein the MiTE polypeptide comprises a binding domain that specifically binds EGFR and/or EGFRvIII and a binding domain that specifically binds CD3.
12. The method of any of the preceding paragraphs, wherein the MiTE polypeptide comprises a binding domain that specifically binds IL13Rα2 and a binding domain that specifically binds CD3.
13. The method of any of the preceding paragraphs, wherein the administering comprises administering a stem cell engineered to express a first and a second MiTE polypeptide.
14. The method of any of the preceding paragraphs, wherein the first MiTE polypeptide comprises a binding domain that specifically binds EGFR and/or EGFRvIII, and a binding domain that specifically binds CD3,
   and the second MiTE polypeptide comprises a binding domain that specifically binds IL13Rα2 and a binding domain that specifically binds CD3.
15. The method of any of the preceding paragraphs, wherein the binding domain that specifically binds CD3 specifically binds to CD38.
16. The method of any of the preceding paragraphs, wherein the binding domains are selected from a nanobody, a single domain antibody, and an scFv.
17. The method of any of the preceding paragraphs, wherein the stem cell is encapsulated in a matrix.
18. The method of any of the preceding paragraphs, wherein the stem cell is administered regionally, locally, or to a tumor resection cavity.
19. The method of any of the preceding paragraphs, wherein the stem cell is further engineered to express an immune modulator polypeptide.
20. The method of any of the preceding paragraphs, further comprising administering a second stem cell population engineered to express an immune modulator polypeptide.
21. The method of any of the preceding paragraphs, wherein the immune modulator is selected from a cytokine and an immune checkpoint inhibitor.
22. The method of any of the preceding paragraphs, wherein the cytokine comprises IL12.
23. The method of any of the preceding paragraphs, wherein the immune checkpoint inhibitor comprises an inhibitor selected from the group consisting of PD-1, PD-L1, TIM-3, LAG-3, CTLA4, or TIGIT.
24. The method of any of the preceding paragraphs, wherein the stem cell is further engineered to express a polypeptide that converts a prodrug to a cytotoxic agent.
25. The method of any of the preceding paragraphs, further comprising administering the prodrug to the subject.
26. The method of any of the preceding paragraphs, wherein the cancer is glioblastoma.
27. The method of any of the preceding paragraphs, wherein the T cell population is administered regionally, locally, or to a site of tumor resection.
28. The method of any of the preceding paragraphs, wherein the T cell population is administered intracerebroventricularly.
29. The method of any of the preceding paragraphs, wherein the stem cells are allogeneic to the subject.
30. The method of any of the preceding paragraphs, wherein the stem cells are autologous to the subject.
31. The method of any of the preceding paragraphs, further comprising, before administering the stem cell, resecting a malignant tumor from the subject.
32. A composition comprising a stem cell engineered to express first and second MiTE polypeptides.
33. The composition of any of the preceding paragraphs, wherein the first MiTE polypeptide comprises a binding domain that specifically binds a first polypeptide expressed on the surface of a cancer cell and a binding domain that specifically binds a polypeptide expressed on the surface of a T cell,
   and the second MiTE polypeptide comprises a binding domain that specifically binds a second polypeptide expressed on the surface of a cancer cell and a binding domain that specifically binds a polypeptide expressed on the surface of a T cell.
34. The composition of any of the preceding paragraphs, wherein the stem cell engineered to express an immune modulator polypeptide is a mesenchymal stem cell (MSC), a neuronal stem cell (NSC) or other stem cell with a tumor tropism.
35. The composition of any of the preceding paragraphs, wherein the engineered stem cell is formulated for delivery to a tumor resection cavity.
36. The composition of any of the preceding paragraphs, wherein the engineered stem cell is encapsulated in a matrix.
37. The composition of any of the preceding paragraphs, wherein the MiTE polypeptide comprises a binding domain that specifically binds EGFR or EGFRvIII, and a binding domain that specifically binds CD3,
   or wherein the MiTE polypeptide comprises a binding domain that specifically binds IL13Rα2 and a binding domain that specifically binds CD3.
38. The composition of any of the preceding paragraphs, wherein the binding domains are selected from a nanobody, a single domain antibody, and an scFv.
39. The composition of any of the preceding paragraphs, wherein the engineered stem cell is further engineered to express an immune modulator polypeptide.
40. The composition of any of the preceding paragraphs, wherein the immune modulator polypeptide is selected from a cytokine and an immune checkpoint inhibitor.
41. The composition of any of the preceding paragraphs, wherein the cytokine comprises IL12.
42. The composition of any of the preceding paragraphs, wherein the immune checkpoint inhibitor comprises an inhibitor of PD-1, PD-L1, TIM-3, LG-3, CTLA4, or TIGIT.
43. The composition of any of the preceding paragraphs, wherein the engineered stem cell is also engineered to express a polypeptide that converts a prodrug to a cytotoxic agent.
44. The composition of any of the preceding paragraphs, further comprising a second stem cell population engineered to express an immune modulator polypeptide.
45. The composition of any of the preceding paragraphs, wherein the immune modulator polypeptide expressed by the second stem cell population is selected from a cytokine and an immune checkpoint inhibitor.
46. The composition of any of the preceding paragraphs, wherein the cytokine comprises IL12.
47. The composition of any of the preceding paragraphs, wherein the immune checkpoint inhibitor comprises an inhibitor of PD-1, PD-L1, TIM-3, LG-3, CTLA4, or TIGIT.
48. A pharmaceutical formulation comprising a composition of any of the preceding paragraphs.
49. A composition comprising a stem cell engineered to express first and second MiTE polypeptides and an isolated population of T cells.
50. A pharmaceutical formulation comprising the composition of any of the preceding paragraphs.
51. A kit comprising a composition of any of the preceding paragraphs, a matrix, a prodrug and packaging materials therefor.
52. The kit of any of the preceding paragraphs, further comprising a second stem cell engineered to express an immune modulator polypeptide.
53. The method of any of the preceding paragraphs, wherein the cancer is a solid tumor.
54. The method of any of the preceding paragraphs, wherein the solid tumor is a primary tumor.
55. The method of any of the preceding paragraphs, wherein the solid tumor is a metastatic tumor.
56. The method of any of the preceding paragraphs, wherein the solid tumor has been resected, or will be resected.
57. A method of treating cancer, the method comprising administering to a subject in need thereof:
   a) an immune cell engineered to secrete a multispecific T cell engager (MiTE) polypeptide construct; and
   b) an isolated population of T cells that are autologous to the subject.
58. The method of any of the preceding paragraphs, wherein the immune cell is a macrophage or a dendritic cell.
59. A composition comprising an immune cell engineered to express first and second MiTE polypeptides.
60. The composition of any of the preceding paragraphs, wherein the immune cell is a macrophage or a dendritic cell.
61. A pharmaceutical formulation comprising the composition of any of the preceding paragraphs.

### EXAMPLES

### Example 1

Engineered T cell-based therapies have demonstrated impressive clinical efficacy in hematological cancers; however, their success has been limited in solid tumors like GBM, particularly due to evasive and inhibitive TME and chronic antigen triggering in the presence of suppressive signals in the TME. Taking advantage of the homing and allorecognition avoidance properties of MSC, we have engineered MSC to release bi-specific T cell engager (BITEs) (this is a MiTE) consisting of nanobodies (vHH domain of a heavy chain antibody) simultaneously targeting over-expressed EGFR and specifically expressed EGFRvIII variant in GBM and a CD3 specific scFv (MSC-ENb-BiTE) (ERGRvIII ab on one end, T cell on the other). Our data indicate that MSC-ENb-BiTE redirect wild type blood derived untransduced T (Tu) cells to tumors and induce tumor regression in GBM mouse models. In order to target multiple antigens and simultaneously enhance the immunomodulatory function of Tu cells in mouse tumor models that mimic clinical settings of GBM tumors we have developed a broad platform of MSC releasing BiTEs targeting EGFR/EGFRvIII and IL13Rα2 and twin (Tw)-BiTE that targets EGFR/EGFRvIII and IL13Rα2 and extensively test their therapeutic efficacy post transplantation of sECM encapsulated MSC-BiTE/Tw-BiTE and intraventricular delivery of Tu cells in mouse GBM models of resection. Based on the hypothesis that interleukin (IL)-12 will improve the potency of T cell therapy and blocking PD-1 on recruited T cells will result in effective eradication of residual GBM cells, we will co-engineer MSC-Tw-BiTE to express IL-12 and immune-check point inhibitor and assess their efficacy with Tu cells in GBM models of resection derived from GBM stem cell (GSC) lines representing distinct tumor nodular and invasive phenotypes. The integration of the safety kill switch in engineered MSC will permits safety of our approach and the incorporation of genetically engineered imaging markers into both MSC and GBMs will allow us to follow fate and efficacy in vivo and thus to fine tune the proposed approaches. The present technology contributes to the development of novel cellular therapies for GBM and defines a new treatment paradigm for patients with other cancers.

### Example 2

### Introduction

Glioblastoma (GBM) is the most common primary brain tumor in adults, characterized by a median survival of 12-14 months with less than 10% of patients surviving at least 2 years from diagnosis [1]. Primary treatment includes surgical resection, chemotherapy and radiation therapy often provided in combination. Among the major challenges associated with GBM treatment, a fundamental role is played by its molecular, genetic and morphological heterogeneity [2].

The discovery of tumor-associated antigens (TAAs) permitted development of a new array of therapeutic methods that selectively target gliomas and spare non-diseased cells of the CNS [3]. In particular, IL13Rα2 is overexpressed in up to 75% of glioma patients [4] and is one of the most thoroughly studied TAA in glioblastoma research since its discovery over two decades ago [5]. Moreover, another main target for GBM therapy is EGFR and its variant EGFRvIII, which are not detected in normal brain but amplified in about 40% of GBM [6]. Among TAA-targeted therapies, those involving the direct engineering of immune cells, demonstrated a remarkable success especially against hematological malignancies. However, their efficacy in solid tumors, and GBM in particular, is still limited and the manufacturing process long and expensive [7].

In detail, a significant hindrance to a successful targeted therapy is represented by the heterogeneous expression of the TAA on the tumor cells and its loss during the treatment, which is associated with therapeutic resistance and tumor recurrence [2, 8]. In this setting, simultaneous targeting of different antigens could enhance targeted therapies by making loss of antigens and tumor escape less likely. Moreover, the efficacy of cell therapies in solid tumors is hindered by the immune suppressive action of the tumor microenvironment (TME) which plays a fundamental role in mitigating anti-tumoral immunity. This is especially true for GBM, also in consideration of the unique immunological characteristics of the brain [9]. Finally, brain cancer is mostly diagnosed at a late stage, thus requiring a timely intervention, possibly at the same time of the resection surgery. A demand incompatible with the time needed to generate patient-derived engineered cells.

As evident from these considerations and from the clinical experience, GBM therapy is in great need of a multi-faceted therapy able to neutralize the several mechanisms protecting the tumor. Data presented herein show an alternative, off-the-shelf approach to activate the immune system and direct it against the tumor, taking into account antigen heterogeneity and TME immune suppression without the need of engineering the patient's immune cells.

A tool to concretize the multiple targeting, is represented by bispecific antibodies called BiTEs (Bispecific T-cell Engagers). In their most common form, those constructs are constituted by two single chain variable fragments, joined to form a link between T-cells and target tumor cells. In fact, one CD3-specific binding arm allows the monovalent binding to all T-cells while the TAA-specific second arm grants the binding to the target cell [10], bringing the effector cell in close proximity with its target. BiTEs used in the past decades showed a high potency of redirected lysis in vitro, and high antitumor activity in various preclinical and clinical settings [11].

IL 13Rα2- and EGFR-targeting BiTEs and engineered mesenchymal stem cells to secrete them (Twin BiTE SC) were generated. The approach was tested on established and primary patient-derived cell lines expressing various levels of the target antigens. The EGFR-binding domain of the BiTE was designed based on the EGFR-specific nanobody generated and characterized by the inventors which significantly reduced GBM growth and invasion in both established and primary invasive GBM in mice [12]. For both BiTEs, the linked CD3-specific domain mirrors the sequence of the CD19-specific BiTE Blinatumomab, approved for clinical use (DrugBank DB09052).

Moreover, to further support the therapeutic action of T cells. Twin BiTE were combined with the immune-stimulating cytokine IL12. In this way, one side the BiTEs activate the lymphocytes against the tumor, while on the other. IL12 sustain the cytotoxic action by counteracting the immune-suppressing microenvironment and preventing exhaustion.

The approach was tested in vivo in orthotopic models of GBM resection that somehow are closer to the clinical scenario of a GBM patient. In this setting, the therapeutic SC were administered by means of a biocompatible gel that concomitantly acts as a scaffold while allowing the cells to release their therapeutic cargo.

### Results

### IL13 and EGFR BiTEs Specifically Eradicate GBM Cells In Vitro

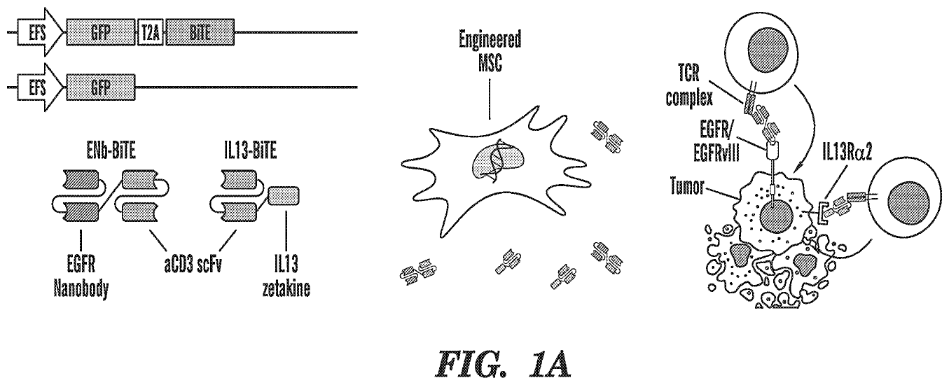

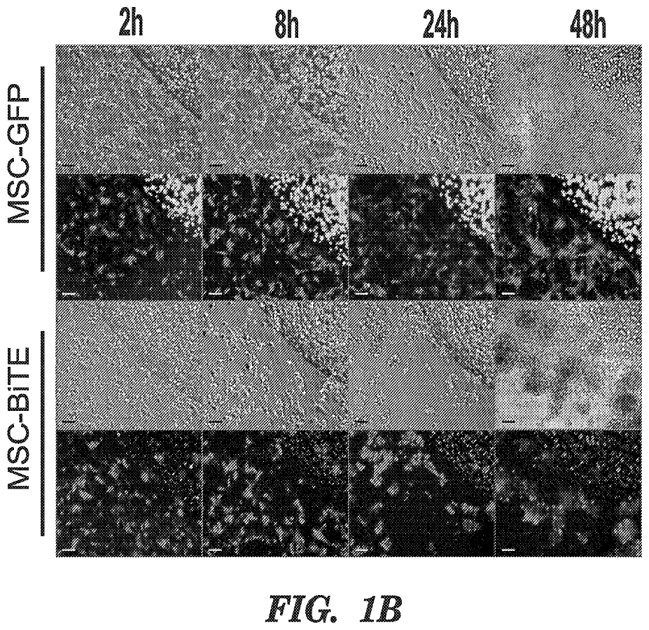

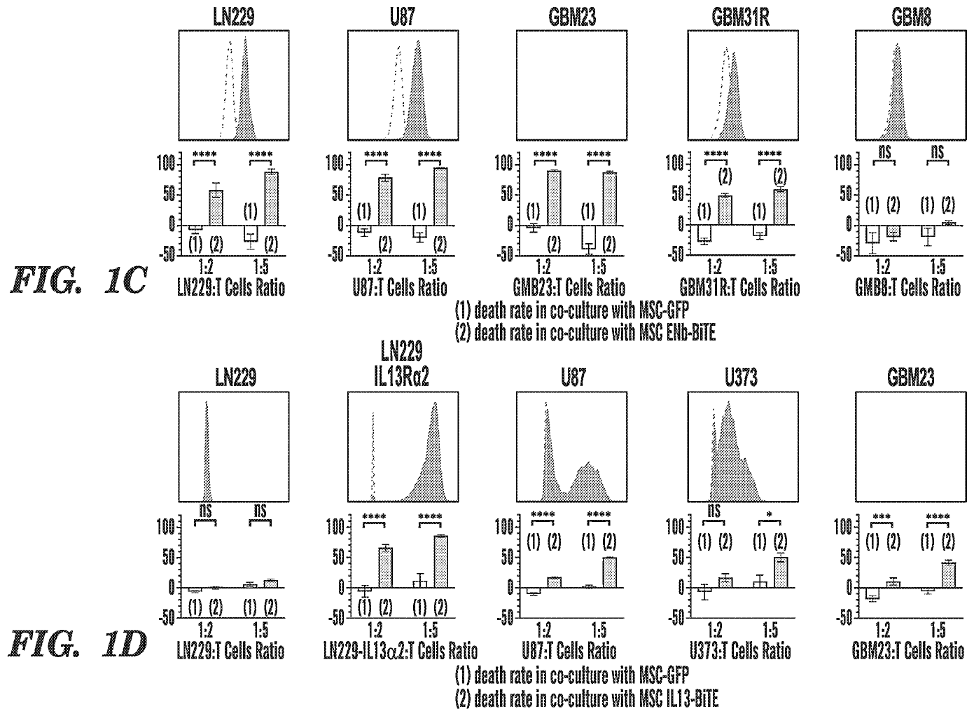

EGFR/EGFRvIII targeting BiTE (ENb-BiTE) were generated by fusing the biparaptopic nanobody specifically binding these antigens and previously described by the inventors with a anti-human CD3 scFv derived from Blinatumumab by means of a serine-glycine linker. Similarly, for the generation of IL13Rα2-specific BiTE (IL13-BiTE) the IL13 ligand was fused to the anti-human CD3 scFv (FIG. 1A). The genes encoding the two BiTEs were cloned into a third-generation lentiviral scaffold and packaged into a lentivirus for stem cell (SC) infection. In vitro, the coculture of tumor cells. T cells and SC-BiTE showed the dynamic of the killing effect. In particular, in the presence of the SC producing the BiTE, the T cells were directed against the tumor cells and eventually resulted in killing them. On the contrary, the tumor cells were left undisturbed when co-cultured with control SC and T cells, showing the specificity and selectivity of the approach (FIG. 1B). Next, this approach was tested in different primary and established GBM cell lines expressing varying levels of EGFR or IL13Ra2 to exemplify the heterogeneity of this tumor. Cytotoxicity assays indicated that a higher level of target antigen expression favors faster killing of the tumor cells (FIG. 1C-1D). For LN229 GBM cells engineered to expressing EGFR at high levels, the death rate after 24 h was >50% at 1:2 Tumor:T cells ratio and 90% at 1:5. A similar high efficacy was observed for U87 GBM cells which also express high levels of EGFR. The death rate was 80% at 1:2 ratio and almost 100% at 1:5. Among the primary patient-derived GBM lines, GBM23 was sensitive to the treatment (90% at 1:2 and 1:5) while. GBM31R characterized by an intermediate expression of EGFR, reached 50% cell death in the two conditions. Finally, the EGFR negative primary cell line GBM8 demonstrated resistance to the treatment, once again showing the specificity of the targeting (FIG. 1C). Furthermore, LN229, which do not express IL13Ra2, were unaffected in co-culture with the corresponding BiTE, but become extremely sensitive when transduced to overexpress IL13Ra2, with 70% cell death at 1:2 ratio and 80% at 1:5. U87 cells, which express high levels of IL13Ra2 in about half their cells, showed a 20% killing at 1:2 ratio with T cells and 50% at 1:5. U373 GBM cells, characterized by a moderate expression, showed 20% cell death at 1:2 and 50% at 1:5 ratio, similar to the primary cell line GBM23 (10% and 40% respectively) (FIG. 1D). These findings indicate that IL13 and EGFR BiTEs released by stem cells specifically eradicate GBM cells in vitro.

### SCs Expressing BiTEs Induce Tumor Regression in Orthotopic Models of GBM.

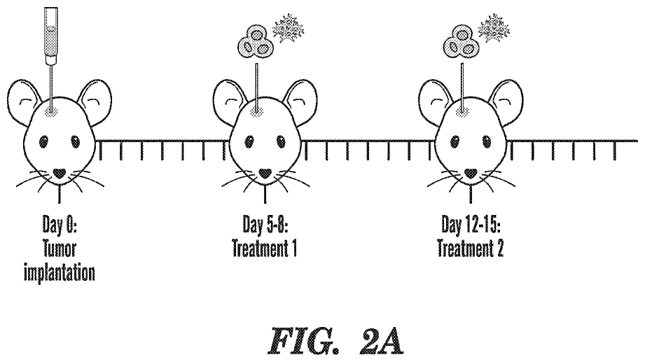

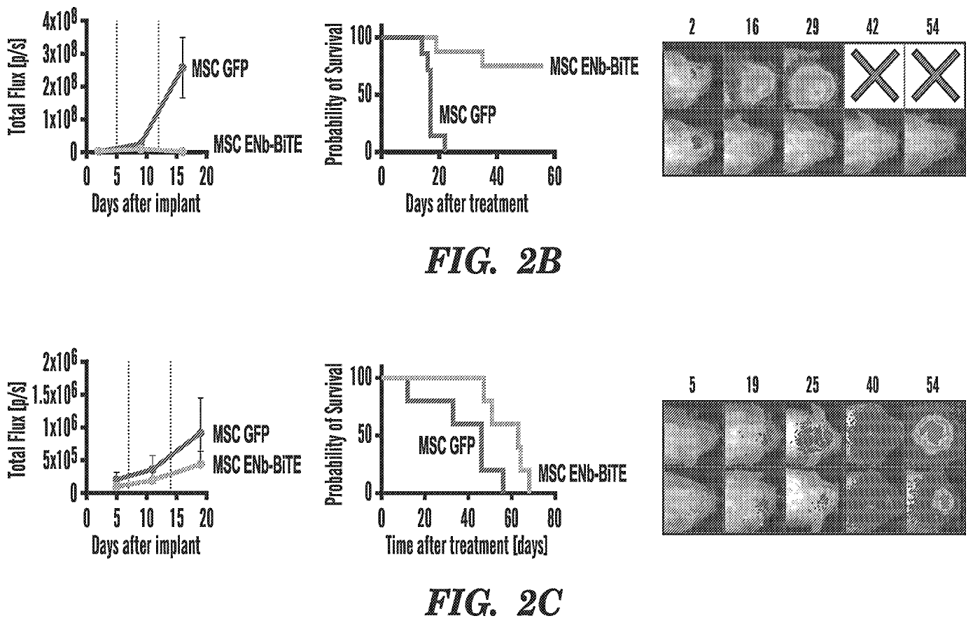

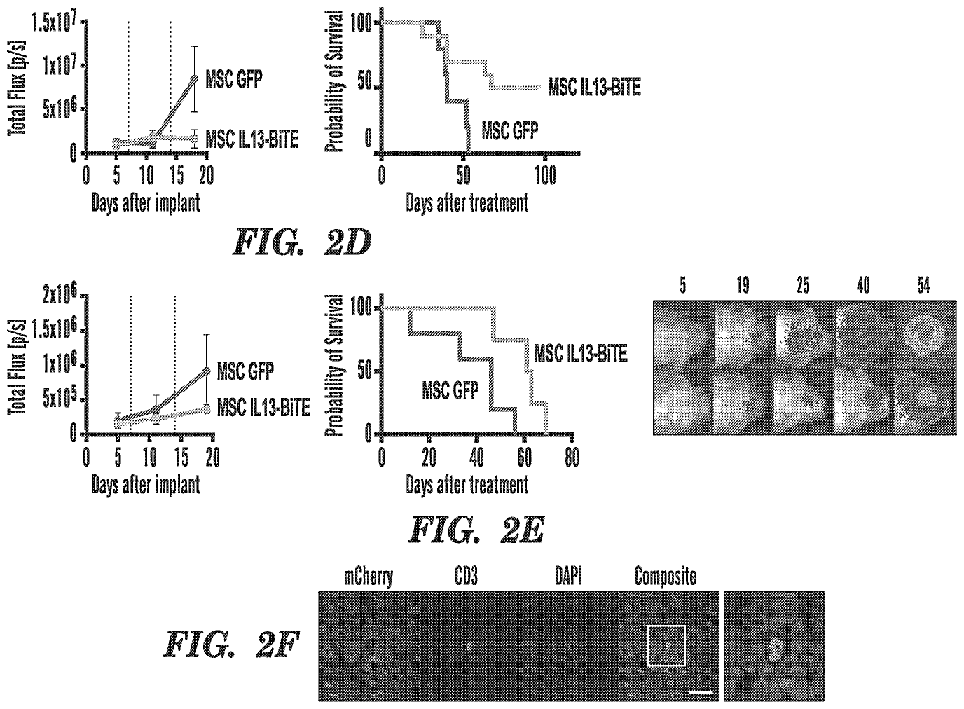

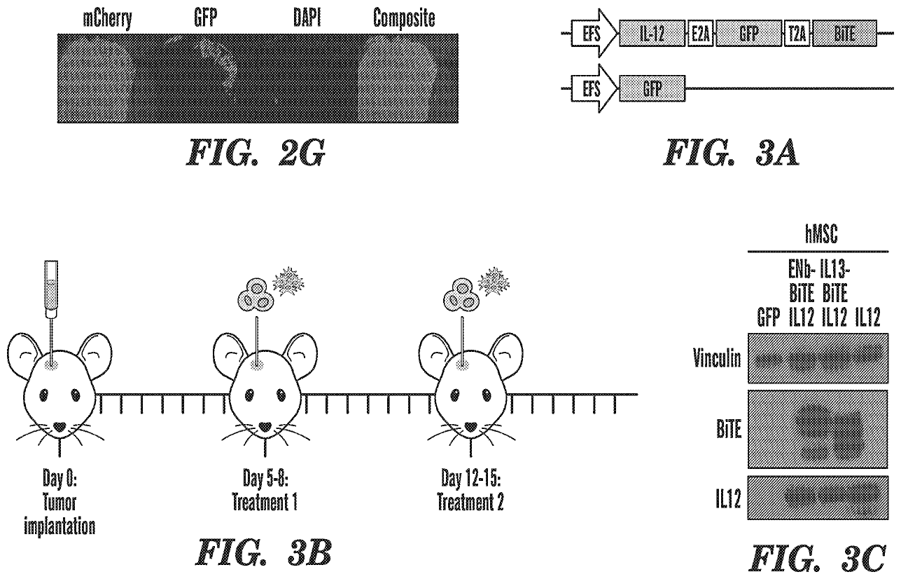

To evaluate the therapeutic efficacy of the individual BiTEs, several animal models were established by implanting tumor cells with different characteristics in the cerebral cortex of NOD/SCID mice and treated with an intra-tumoral injection of SC secreting BiTE and T cells (FIG. 2A). U87 cells were chosen as a model for their high EGFR expression to test SC-ENb-BiTE, which led to a sustained tumor regression end eradication in the treated group (FIG. 2B). For analogue reason, LN229-IL13Rα2 were used as the model to test SC IL13-BiTE, extending the mice lifespan and remarkably reducing the tumor size (FIG. 2D). The model also included GBM23 cells which has a heterogeneous expression of the two TAAs, EGFR and IL13Rα2. In this case, the individual BiTEs failed to fully eradicate the tumor, although prolonging mice survival and contributing to tumor reduction. It is specifically hypothesized that the heterogeneous expression of the target antigen, allowed the tumor to escape the T cell killing and recur (FIGS. 2C and 2E). The presence of T cells and SC in or around the tumor was confirmed by immune fluorescence (FIG. 2F-2G). These findings confirmed that SCs expressing BiTEs and T cells induce tumor regression in orthotopic models of GBM.

### The Combination With IL-12 Reduces Exhaustion and Improves Survival In Vivo.

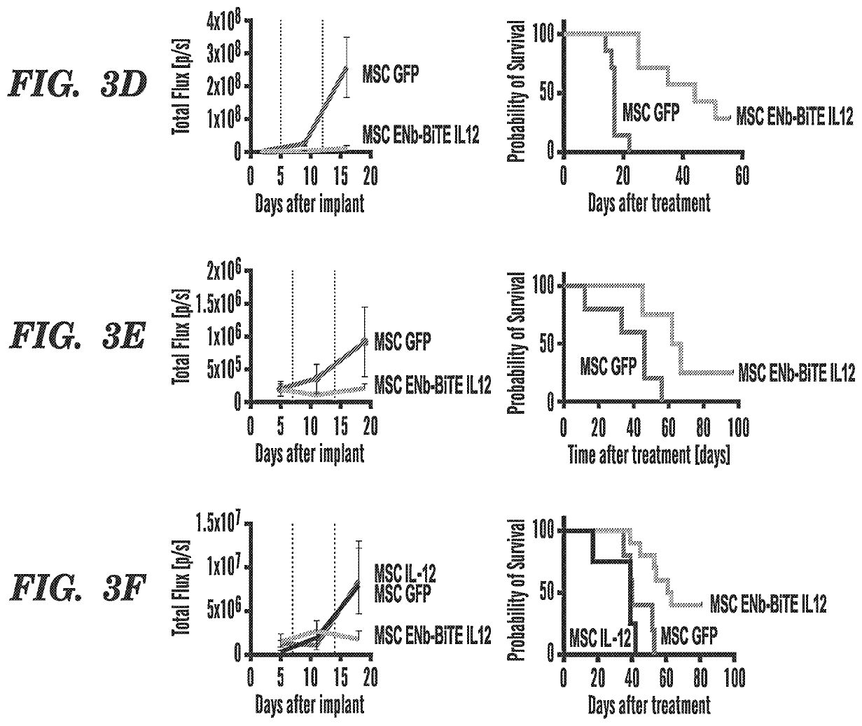

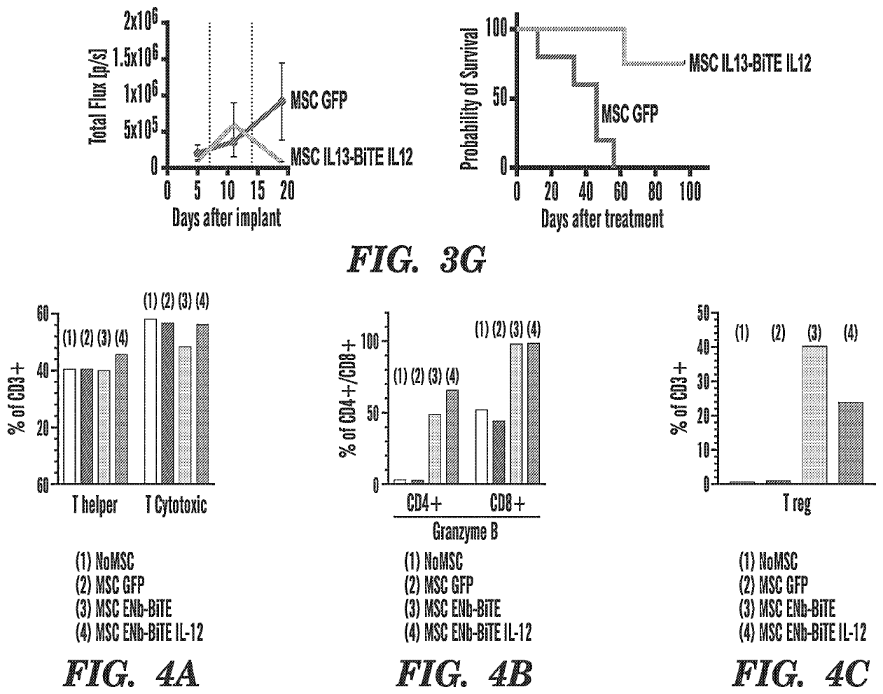

Next, SC to co-express BiTEs and IL-12 were created and tested the combination with IL-12 secretion and SC-BiTEs/T cells in U87, LN229-IL13Rα2 and GBM23 tumor models. A tangible benefit was observed in particular for the GBM23 model, with an extension of survival and smaller tumors (FIG. 3A-3F).

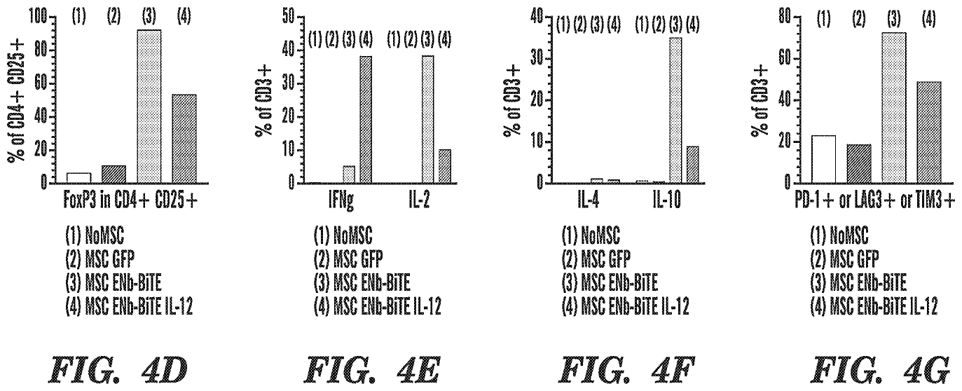

To investigate the possible effect of IL-12 on T cells in the setting of a BiTE-driven therapy, T cells were characterized after co-culture with tumor cells in the presence of SC secreting BiTEs or control by flow cytometry. In particular, the comparison of the subpopulations of T cells in presence of IL-12 showed a decrease in T regs, likely due to a reduction of FOXP3 expression, without affecting the distribution of CD4 and CD8 cells (FIGS. 4A, 4C and 4D). At the same time, cytotoxicity markers, such as Granzyme B, were upregulated by the BiTE-mediated activation in both CD4 and CD8 cells (FIG. 4B). IL-12 also boosted the production of IFNg, overall shaping a Th1 phenotype, corroborated by the low level of IL-4 and the IL12-mediated reduction of IL-10 (FIG. 4E-4F). Finally, IL-12 showed a favorable effect in reducing the overall expression of exhaustion markers PD-1, LAG-3, TIM-3 (FIG. 4G). These findings indicated that the combination of SC-BITES/T cells and IL-12 reduces exhaustion and improves survival in vivo.

### The Twin BiTE Extends Mice Survival in Heterogeneous Primary Tumor Model.

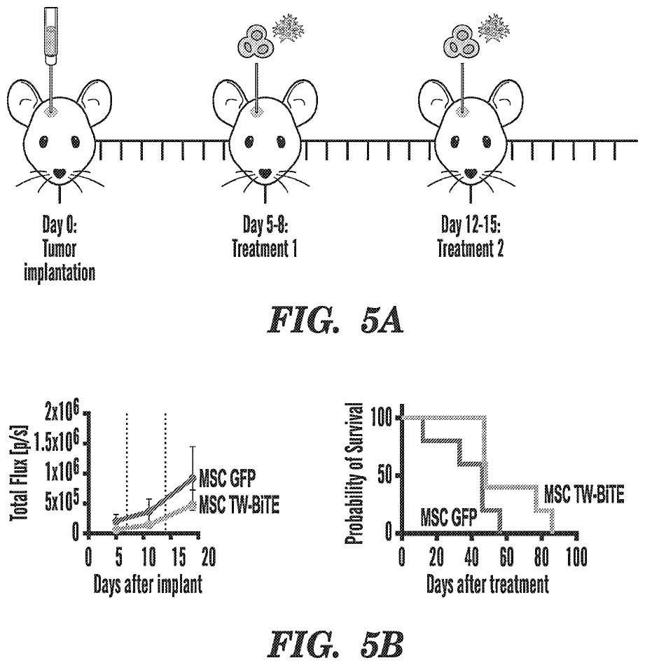

The intra-tumoral administration of Twin BiTE SC provided a therapeutic benefit in the heterogeneous GBM23 model by prolonging the survival in the treated mice compared to the control and the single BiTEs (FIGS. 5A and 5B).

### Summary

The in vitro and in vivo data presented herein show the efficacy of the individual BiTEs against different primary and established tumor cell lines. Moreover, the Twin BiTE SC significantly controlled the growth of a tumor expressing heterogeneous levels of both IL13Ra2 and EGFR. The combination with IL12 reduced T cell exhaustion and consolidated a Th1 phenotype. The orthotopic resection model showed the feasibility of the approach in a clinically-relevant setting. Data presented herein demonstrate the feasibility and efficacy of an off-the-shelf immunotherapy, based on BiTEs simultaneously targeting tumor heterogeneity without the need for direct engineering of the immune cells. The clinically-relevant model further supports the translatability of the approach which would be performed at the time of surgery, with great benefits for the patients.

### REFERENCES

1. Gately L, Mclachlan S, Dowling A, Philip J. Life beyond a diagnosis of glioblastoma: a systematic review of the literature. J Cancer Surviv. 2017; 11:447-52.
2. Sharma P. Debinski W. Receptor-Targeted Glial Brain Tumor Therapies. Int J Mol Sci. 2018; 19:3326.
3. Malpass K. Identification of novel glioblastoma-associated antigens reveals targets for immunotherapy. Nat Rev Neurol. 2012; 8:240-240.
4. Sattiraju A, Sai KKS, Xuan A, Pandya DN, Almaguel FG, Wadas TJ, et al. IL13RA2 targeted alpha particle therapy against glioblastomas. Oncotarget. 2017; 8.
5. Debinski W, Obiri NI, Powers SK, Pastan I, Puri RK. Human glioma cells overexpress receptors for interleukin 13 and are extremely sensitive to a novel chimeric protein composed of interleukin 13 and Pseudomonas exotoxin. Clin Cancer Res Off J Am Assoc Cancer Res. 1995; 1:1253-8.
6. Gan HK, Kaye AH, Luwor RB. The EGFRvIII variant in glioblastoma multiforme. J Clin Neurosci. 2009; 16:748-54.
7. Charrot S, Hallam S. CAR-T Cells: Future Perspectives. HemaSphere. 2019; 3:e188.
8. Brown CE, Alizadeh D, Starr R, Weng L, Wagner JR, Naranjo A, et al. Regression of Glioblastoma after Chimeric Antigen Receptor T-Cell Therapy. N Engl J Med. 2016; 375:2561-9.
9. Desland FA, Hormigo A. The CNS and the Brain Tumor Microenvironment: Implications for Glioblastoma Immunotherapy. Int J Mol Sci. 2020; 21:7358.
10. Mack M, Riethmuller G, Kufer P. A small bispecific antibody construct expressed as a functional single-chain molecule with high tumor cell cytotoxicity. Proc Natl Acad Sci. 1995; 92:7021-5.
11. Baeuerle PA, Reinhardt C. Bispecific T-Cell Engaging Antibodies for Cancer Therapy. Cancer Res. 2009; 69:4941-4.
12. van de Water JAJM, Bagci-Onder T, Agarwal AS, Wakimoto H, Roovers RC, Zhu Y, et al. Therapeutic stem cells expressing variants of EGFR-specific nanobodies have antitumor effects. Proc Natl Acad Sci. 2012; 109:16642-7.
13. Stern LA, Gholamin S, Moraga I, Yang X, Saravanakumar S, Cohen JR, et al. Engineered IL13 variants direct specificity of IL13Rα2-targeted CAR T cell therapy. Proc Natl Acad Sci. 2022; 119:e2112006119.

What is claimed is:

1. A method of treating cancer, the method comprising administering to a subject in need thereof:
a) a stem cell or immune cell engineered to secrete a multispecific T cell engager (MITE) polypeptide construct; and
b) an isolated population of T cells that are autologous to the subject.

2. The method of claim 1, wherein the stem cell has a tumor tropism.

3. The method of claim 1, wherein the stem cell is a mesenchymal stem cell (MSC) or a neuronal stem cell (NSC).

4. The method of claim 1, wherein the stem cell is at least one of:
a) derived from an induced pluripotent stem cell,
b) encapsulated in a matrix,
c) allogeneic to the subject,
d) autologous to the subject,
e) further engineered to express an immune modulator polypeptide, and
f) further engineered to express a polypeptide that converts a prodrug to a cytotoxic agent.

5. The method of claim 1, wherein the isolated population of T cells is at least one of:
a) enriched for gd T cells;
b) not genetically manipulated; and
c) expanded in culture prior to administering.

6-7. (canceled)

8. The method of claim 1, wherein the MiTE polypeptide comprises a binding domain that specifically binds a polypeptide expressed on the surface of a cancer cell and a binding domain that specifically binds a polypeptide expressed on the surface of a T cell.

9. The method of claim 8, wherein the polypeptide expressed on the surface of a cancer cell is selected from the group consisting of EGFR, EGFRvIII, HER2, IL-13R 2, EphA2 and GD2, or
wherein the polypeptide expressed on the surface of a T cell is selected from the group consisting of CD3, CD28, 41BB and OX40.

10.-12. (canceled)

13. The method of claim 1, wherein the administering comprises administering a stem cell engineered to express a first and a second MiTE polypeptide.

14. The method of claim 13, wherein the first MiTE polypeptide comprises a binding domain that specifically binds EGFR and/or EGFRvIII, and a binding domain that specifically binds CD3,
and the second MiTE polypeptide comprises a binding domain that specifically binds IL13Rα2 and a binding domain that specifically binds CD3.

15.-17. (canceled)

18. The method of claim 1, wherein the stem cell is administered regionally, locally, or to a tumor resection cavity.

19. (canceled)

20. The method of claim 1, further comprising administering a second stem cell population engineered to express an immune modulator polypeptide.

21. The method of claim 20, wherein the immune modulator is selected from a cytokine and an immune checkpoint inhibitor.

22.-25. (canceled)

26. The method of claim 1, wherein the cancer is glioblastoma.

27. The method of claim 1, wherein the T cell population is administered regionally, locally, or to a site of tumor resection, or wherein the T cell population is administered intracerebroventricularly.

28.-30. (canceled)

31. The method of claim 1, further comprising, before administering the stem cell, resecting a malignant tumor from the subject.

32. A composition comprising a stem cell engineered to express first and second MiTE polypeptides.

33. The composition of claim 32, wherein the first MiTE polypeptide comprises a binding domain that specifically binds a first polypeptide expressed on the surface of a cancer cell and a binding domain that specifically binds a polypeptide expressed on the surface of a T cell,
and the second MiTE polypeptide comprises a binding domain that specifically binds a second polypeptide expressed on the surface of a cancer cell and a binding domain that specifically binds a polypeptide expressed on the surface of a T cell.

34.-38. (canceled)

39. The composition of claim 32, wherein the engineered stem cell is further engineered to express an immune modulator polypeptide, and/or wherein the engineered stem cell is also engineered to express a polypeptide that converts a prodrug to a cytotoxic agent.

40.-48. (canceled)

49. A composition comprising a stem cell engineered to express first and second MiTE polypeptides and an isolated population of T cells.

50.-57. (canceled)

58. The method of claim 1, wherein the immune cell is a macrophage or a dendritic cell.

59.-61. (canceled)
